(19) United States
(12) Patent Application Publication
(10) Pub. No.: US 2026/0284116 A1
(43) Pub. Date: Sep. 24, 2026

(54) METHOD OF TREATING INFLAMMATION WITH CELLULAR COMPOSITIONS

(71) Applicant: MESOBLAST INTERNATIONAL SARL, Meyrin (CH)

(72) Inventors: Silviu ITESCU, Melbourne (AU); Paul SIMMONS, Melbourne (AU); Jack HAYES, New York, NY (US); Justin HORST, New York, NY (US); Fiona SEE, Melbourne (AU)

(73) Assignee: MESOBLAST INTERNATIONAL SARL, Meyrin (CH)

(21) Appl. No.: 19/136,773

(22) PCT Filed: Dec. 8, 2023
(86) PCT No.: PCT/IB2023/062428

§ 371 Date: Jun. 6, 2025

Related U.S. Application Data
(60) Provisional application No. 63/386,876, filed on Dec. 9, 2022.
(60) Provisional application No. 63/386,883, filed on Dec. 9, 2022.
(60) Provisional application No. 63/507,009, filed on Jun. 8, 2023.

(51) Int. Cl. A61K 35/28; A61P 37/06

(52) U.S. Cl. CPC A61K 35/28; A61P 37/06

(57) ABSTRACT

The present disclosure relates to cellular compositions with anti-inflammatory properties and use of the same in methods for treating inflammation.

### TECHNICAL FIELD

The present disclosure relates to cellular compositions with anti-inflammatory properties and use of the same in methods for treating inflammation.

### BACKGROUND

Inflammation is a defensive host response to a variety of stimuli including foreign materials (e.g. microbes), trauma, malignancy, and/or toxins. Inflammation is a complex and multi-faceted response that is broadly regulated by upregulation of pro-inflammatory cytokines which leads to the accumulation and activation of leukocytes. Excessive or persistent inflammation can lead to, or occur as a result of, inflammatory diseases such as diabetes, graft versus host disease, chronic pain, cancer, as well as infectious or post-infectious diseases.

Despite there being an array of drugs on the market for treating inflammation, inflammatory disease affects millions of people worldwide every year and is associated with high rates of morbidity and mortality. Thus, there is a need in the art for improved treatments for inflammation and inflammatory disease.

### SUMMARY

The present inventors have surprisingly identified that mesenchymal lineage precursor or stem cells (MLPSCs) which have been culture expanded in a culture medium comprising non-fetal serum are particularly effective in treating subjects with persistent inflammation.

Accordingly, in an example, the present disclosure relates to a method of treating inflammation in a subject, the method comprising administering to the subject a composition comprising a population of culture expanded mesenchymal lineage precursor or stem cells (MLPSCs), wherein the MLPSCs have been culture expanded in a cell culture medium comprising at least one pro-inflammatory cytokine. In an example, the pro-inflammatory cytokine is selected from the group consisting of IL-1β, IL-6, TNF-α, IFN-γ and/or IL-1RA. In an example, the pro-inflammatory cytokine is provided in a non-fetal serum. Therefore, in an example, the cell culture medium comprises non-fetal serum.

Accordingly, in an example, the disclosure also provides a method of treating inflammation in a subject, the method comprising administering to the subject a composition comprising a population of culture expanded mesenchymal lineage precursor or stem cells (MLPSCs), wherein the MLPSCs have been culture expanded in a cell culture medium comprising non-fetal serum.

In an example, the subject has persistent inflammation.

In an example, the non-fetal serum is new born calf serum (NBCS). In an example, the NBCS is obtained≤30 after birth of the calf. In an example, the NBCS is obtained≤21 after birth of the calf. In an example, the NBCS is obtained between the day of birth and 21 days after birth of the calf. In an example, the NBCS is obtained between the day of birth and 14 days after birth of the calf. In an example, the NBCS is obtained between the day of birth and 10 days after birth of the calf. In an example, the NBCS is obtained between the day of birth and 7 days after birth of the calf. In an example, the NBCS is obtained between the day of birth and 4 days after birth of the calf. In an example, the NBCS is obtained between the day of birth and 3 days after birth of the calf. In an example, the NBCS is obtained after the calf has received colostrum.

In an example, the non-fetal serum is characterised by the presence of one or more pro-inflammatory cytokines. In an example, the non-fetal serum is characterised by the presence of any one or more of IL-1β, IL-6, TNF-α, IFN-γ and/or IL-IRA.

In an example, methods of the disclosure comprise treating an inflammatory disease in a subject.

In an example, the inflammatory disease is refractory to steroid immunosuppressant and/or a biologic therapy,

In an example, the inflammatory disease is mediated by T-cell activation and/or proliferation.

In an example, the inflammatory disease is refractory to steroid immunosuppressant and/or ruxolitinib. In an example, the inflammatory disease is refractory to steroid immunosuppressant and ruxolitinib. In an example, the inflammatory disease is GvHD and the GvHD is refractory to steroid immunosuppressant and ruxolitinib.

In an example, the inflammatory disease is hyperinflammation. In an example, the hyperinflammation is caused by a coronavirus infection. For example, the coronavirus is coronavirus (SARS-COV), Middle East Respiratory Syndrome coronavirus (MERS-COV) or COVID-19.

In an example, the inflammatory disease is multi-system inflammatory syndrome (MIS). In an example, the inflammatory disease is acute respiratory distress syndrome (ARDS).

In an example, the inflammatory disease is selected from the group consisting of graft versus host disease (GvHD), pruritus, skin inflammation, psoriasis, multiple sclerosis, rheumatoid arthritis, osteoarthritis, systemic lupus erythematosus, Hashimoto's thyroidis, myasthenia gravis, type I or II diabetes, diabetic nephropathy, asthma, inflammatory lung injury, inflammatory liver injury, inflammatory glomerular injury, atopic dermatitis, allergic contact dermatitis, irritant contact dermatitis, seborrhoeic dermatitis, Sjoegren's syndrome, keratoconjunctivitis, uveitis, inflammatory bowel disease, Crohn's disease, ulcerative colitis, an inflammatory disease of the joints, skin, or muscle, acute or chronic idiopathic inflammatory arthritis, myositis, a demyelinating disease, chronic obstructive pulmonary disease (COPD), interstitial lung disease, interstitial nephritis and chronic active hepatitis.

In an example, the inflammatory disease is diabetes or an associated condition or symptom of diabetes selected from the group consisting of abnormal wound healing, kidney failure, blindness, neuropathy, nephropathy, retinopathy, inflammation, impotence or nonalcoholic steatohepatitis (NASH). In an example, the inflammatory disease is type II diabetes. In an example, the inflammatory disease is rheumatoid arthritis. In an example, the inflammatory disease is diabetic nephropathy.

In an example, the inflammatory disease is pain. In an example, the pain is chronic pain. In an example, the inflammatory disease may be lower back pain. In an example, the inflammatory disease is lower back pain that is associated with a degenerated disc.

In an example, the MLPSCs are administered systemically. In an example, the MLPSCs are administered intravenously. In an example, the MLPSCs are administered over multiple doses. In an example, between 1×10⁷ and 2×10⁸ cells are administered.

In an example, the MLPSCs are mesenchymal precursor cells (MPCs). In an example, the MPCs are isolated from bone mononuclear cells with an anti-STRO-3 antibody before culture expansion.

In an example, the mesenchymal lineage precursor or stem cells are mesenchymal stem cells (MSCs).

In an example, the MLPSCs are allogeneic.

In an example, the cells have been cryopreserved prior to administration. In an example, the cells have been cryopreserved prior to culture expansion in a cell culture medium comprising non-fetal serum.

In an example, the population of MLPSCs is provided in a composition which comprises Plasma-Lyte A, dimethyl sulfoxide (DMSO), human serum albumin (HSA). In an example, the composition comprises Plasma-Lyte A (70%), DMSO (10%), HSA (25%) solution, the HSA solution comprising 5% HSA and 15% buffer. In an example, the composition comprises greater than 6.68×10⁶ viable cells/mL.

In an example, the MLPSCs have been culture expanded in a cell factory. In an example, the cell factory is passively primed with CO₂ before seeding cells.

In an example, the method comprising selecting for use in treatment culture expanded MLPSCs that have increased level(s) of one or more angiogenic markers selected from the group consisting of:
the level of VEGF, angiogenin and/or SDF-1α expressed by the MLPSCs under culture conditions; and/or,
the level of endothelial network formation, endothelial network length, endothelial branch length measured after treating a population of endothelial cells with conditioned media obtained from the MLPSCs.

In an example, an increased level of the one or more angiogenic markers is determined relative to a population of MLPSCs that have been culture expanded in a cell culture medium comprising 10% fetal serum

### BRIEF DESCRIPTION OF DRAWINGS

FIG. 1: Serum cytokine levels assessment and comparison. 1:1 FCS/NBCS (serum A); fetal bovine serum (serum B); FBS from a different supplier (serum C).

FIG. 2: Quantitative measurement of in-vitro angiogenesis induced by MLPSC conditioned media using IncuCyte® 96-Well Kinetic Angiogenesis PrimeKit Assay.

FIG. 3: Luminex assay results showing increased production of angiogenin by MLPSCs cultured in the presence or absence of newborn serum.

FIG. 4: Angiogenic marker levels in MLPSC-conditioned media from cGMP lots cultured in the presence or absence of newborn serum.

FIG. 5: Analysis of Change from Baseline at 12 Months in Echo Parameters
all subjects.

FIG. 6: Analysis of Change from Baseline at 12 Months in Echo Parameters
subjects with persistent inflammation (hsCRP≥2).

FIG. 7: Analysis of Change from Baseline at 12 Months in Echo Parameters
subjects without persistent inflammation (hsCRP<2).

FIG. 8: CV Death in in subjects with persistent inflammation (hsCRP≥2) by MPCs cultured in the presence or absence of newborn serum.

FIG. 9: 3-Point composite MACE (MI, Stroke or CV Death) in subjects with persistent inflammation (hsCRP>2) by MPCs cultured in the presence or absence of newborn serum.

FIG. 10: 3-Point composite MACE (MI, Stroke or CV Death) in subjects administered MPCs cultured in the presence or absence of newborn serum in all or final passages. LHS figure represents data from all patients. RHS figure represents data from patients with persistent inflammation (CRP>2 mg/ml).

FIG. 11: 3-Point composite MACE (LHS figure) and Terminal Cardiac Events (TCE; RHS figure) in subjects with most severe disease (NTpro-BNP>1000 ng/ml; CRP>2 mg/ml) administered MPCs cultured in the presence or absence of newborn serum in all or final passages.

FIG. 12: (A) Pro-inflammatory cytokine IL-6 plasma levels in control LVAD patients: ischemic versus non-ischemic HFrEF etiology. (B) Pro-inflammatory cytokine IL-6 plasma levels in LVAD patients: ischemic controls versus ischemic LVAD patients administered MPCs.

FIG. 13: All-Cause Death over 12 months in ischemic and non-ischemic LVAD control patients.

FIG. 14: All-Cause Death over 12 months in LVAD patients administered “Licensed” MPC formulation, “Unlicensed” MPC formulation, and control patients. (A) All LVAD patients (ischemic and non-ischemic groups). (B) Ischemic LVAD patients.

### DETAILED DESCRIPTION

### General Techniques and Definitions

Unless specifically defined otherwise, all technical and scientific terms used herein shall be taken to have the same meaning as commonly understood by one of ordinary skill in the art (e.g., in cell therapy, molecular biology, stem cell culture, immunology, and biochemistry).

Unless otherwise indicated, cell culture techniques and assays utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more preferably +/−5%, of the designated value.

As used herein, the term “inflammation” in the context of the present disclosure refers to a physiological and/or pathophysiological state characterised by the presence of inflammatory biomarkers, for example pro-inflammatory cytokines and activation of immune cells. “Inflammatory disease” as used herein refers to any disease, disorder, or condition wherein a subject experiences ongoing or persistent inflammation. An “inflammatory disease” according to the disclosure can also include any disease, disorder or condition that is secondary to an acute inflammatory episode.

“Persistent inflammation” is defined by elevated C-reactive protein levels. In an example, persistent inflammation is characterised by CRP levels ≥2 mg/L. “C-reactive protein” or “CRP” is an inflammatory mediator. CRP levels are raised under conditions of acute inflammatory recurrence and rapidly normalize once the inflammation subsides. Accordingly, CRP is an effective marker of persistent inflammation. In an example, subjects treated according to the present disclosure may have elevated CRP. The term “elevated CRP” is used in the context of the present disclosure to refer to CRP levels that are increased relative to baseline CRP levels. In an example, CRP levels ≥1 mg/L are elevated. In another example, CRP levels ≥1.5 mg/L are elevated. In another example, CRP levels ≥2 mg/L are elevated. In another example, CRP levels ≥4 mg/L are elevated.

In an example, the persistent inflammation is persistent systemic inflammation. In this example, the persistent inflammation is characterised by a systemic biomarker of the inflammation such as CRP. Accordingly, in an example, methods of the present disclosure are directed towards treating inflammation characterised by persistent systemic inflammation. In an example, methods of the present disclosure are used to treat an inflammation associated with persistent elevation in IL-6 levels.

The term “angiogenic marker” as used herein refers an indicator of angiogenesis. As used herein, “angiogenic markers” include pro-angiogenic molecules, for example, VEGF, angiogenin, and SDF-1α. In another example, angiogenic markers are cellular indicators of angiogenesis, for example, endothelial network formation, endothelial network length, and endothelial branch length. In this example, cellular indicators of angiogenesis are determined in an in-vitro angiogenesis assay as disclosed herein.

The terms “level” and “amount” are used to define the amount of a particular substance in a sample from a subject or in a cell culture media (or sample therefrom). For example, a particular concentration, weight, percentage (e.g. v/v %) or ratio can be used to define the level of a particular substance in a sample. In an example, the level is expressed in terms of how much of a particular marker is expressed by cells of the disclosure under culture conditions. In an example, expression represents cell surface expression. In another example, the level is expressed in terms of how much of a particular marker is released from cells described herein under culture conditions. In an example, the sample is obtained from a patient or subject (e.g. a blood sample) and the level of a substance is measured in the sample to determine the level of the substance in the sample. For example, a blood sample can be obtained to measure the level of CRP in a subject.

In an example, the level is expressed in mg/L. For example, a level of CRP can be expressed in mg/L. In an example, the level is expressed in ng/ml. For example, a level of VEGF can be expressed in ng/ml. In an example, a level of SDF-1α can be expressed in ng/ml. In an example, the level is expressed in pg/ml. In an example, a level of angiogenin can be expressed in pg/ml.

In an example, the level of a particular marker is measured in a population of cells (or supernatant obtained following cell culture of the same) and divided by the number of cells in the population. In this example, the level may be presented in units (e.g. pg) per 10⁶ cells.

In an example, the level of a particular marker in a cell culture medium is determined under culture conditions. The term “culture conditions” is used to refer to cells growing in culture. In an example, culture conditions refers to an actively dividing population of cells. Such cells may, in an example, be in exponential growth phase. In an example, the cells are in a stationary phase.

In an example, culture conditions encompass co-culture of an MLPSC population disclosed herein and a second cell population such as a population which comprises peripheral blood mononuclear cells (PBMC). In an example, co-culture comprises culturing an MLPSC population disclosed herein and a population of activated PBMC. For example, PBMC can be activated using anti-CD3 and anti-CD28 antibodies before co-culture with an MLPSC population disclosed herein.

In an example, “culture conditions” comprises co-culturing MLPSCs and T cells at a ratio of about 1 MLPSC:2 T cells, or less. For example, 1:3, 1:4, 1:5, 1:10, 1:20, 1:30, 1:40, 1:50, 1:60, 1:70 1:80, 1:90, or 1 MLPSC: 100 T cells, or less. In this example, the level of IL2-RA inhibition is determined after about 30 to 84 hours of cell culture under culture conditions.

In an example, the level of a particular marker can be determined by taking a sample of cell culture media and measuring the level of marker in the sample. In another example, the level of a particular marker can be determined by taking a sample of cells and measuring the level of the marker in the cell lysate. Those of skill in the art will appreciate that secreted markers will generally be measured by sampling the culture media while markers expressed on the surface of the cell may generally be measured by assessing a sample of cell lysate. In an example, the sample is taken when the cells are in exponential growth phase. In an example, the sample is taken after at least two days in culture. In another example, the sample is taken after about 30 to 84 hours of culture. In an example, the sample is taken after culture in cell culture medium comprising non-fetal serum followed by two to three days culture cell culture medium comprising 10% fetal serum.

In an example, the sample is taken from a co-culture of MLPSCs and activated PBMCs. In this example, the cell sample can be lysed and the level of a marker can be determined. For example, the level of IL2-RA may be determined. In his example, the level of IL2-RA can be determined using various methods such as an enzyme-linked immunosorbent assay (ELISA) based method. In an example, the ELISA comprises:
(i) adding sample diluent to each well of a microplate precoated with a monoclonal antibody specific for IL2-RA;
(ii) adding a co-cultured sample to a well of a microplate precoated with a monoclonal antibody specific for IL2-RA;
(iii) incubating the microplate for sufficient time to allow for the monoclonal antibody specific for IL2-RA to specifically bind to any IL2-RA in the sample;
(iv) washing the microplate;
(v) adding IL2-RA conjugate to the well;
(vi) incubating the microplate for sufficient time to allow the conjugate to specifically bind to any captured IL2-RA;
(vii) washing the microplate;
(viii) adding a substrate solution to the well;
(ix) incubating the microplate for sufficient time for colour development;
(x) adding a stop solution to the well;
(xi) reading optical density on a microplate reader set to 450 nm with wavelength correction at 570 nm;
(xii) determining the level of IL2-RA.

In another example, the level of IL2-RA is determined using fluorescence-activated cell sorting (FACS) using appropriate antibodies such as anti-CD25. Further antibodies may also be employed if required to distinguish CD25+ cell types. While the above referenced examples refer to IL-2RA, it will be appreciated that similar methods may also be used to determine the level of other markers disclosed herein such as angiogenin. In these examples, co-culture may not be required to determine the level. For example, the level of angiogenin may be measured in a population of MLPSCs under culture conditions.

Culture expanding cells from a cryopreserved intermediate means thawing cells subject to cryogenic freezing and in vitro culturing under conditions suitable for growth of the cells.

In an example, the “level” or “amount” of a particular marker is determined after cells have been cryopreserved and then seeded back into culture. For example, the level may be determined after a first cryopreservation of cells. In another example, the level is determined after a second cryopreservation of cells. In an example, cells are isolated from an appropriate stem cell source such as bone marrow (e.g. using immune-selection for marker(s) such as STRO-1), culture expanded to provide an intermediate cell population and assessed to determine the level of a particular marker. In this example, the level may be determined before or after cryopreservation. In an example, the level is determined after cryopreservation of the intermediate cell population. In another example, cells may be culture expanded from a cryopreserved intermediate, cryopreserved a second time before being re-seeded in culture so that the level of a particular marker can be determined under culture conditions.

As used herein, the terms “treating”, “treat”, “treatment”, “reducing progression” include administering a population of MLPSCs cultured according to the present disclosure and/or progeny thereof and/or soluble factors derived therefrom and/or extracellular vesicles derived therefrom to thereby reduce or eliminate at least one symptom of inflammation or, in the context of reducing progression, delay development of the same.

In an example, treatment reduces the level of one or more of inflammatory biomarkers, for example neutrophilia, lymphopenia, thrombocytopenia, hypoalbuminemia, elevated CRP, erythrocyte sedimentation rate (ESR), fibrinogen, D-dimer, ferritin, lactic acid dehydrogenase (LDH), interleukin 6 (IL-6), elevated procalcitonin. In an example, treatment reduces the level of white blood cell levels, triglyceride levels, CRP or IL-6. In an example, treatment reduces the level of one or more pro-inflammatory cytokine.

The term “subject” as used herein refers to a human subject. For example, the subject can be an adult. In another example, the subject can be a child. In another example, the subject can be an adolescent. Terms such as “subject”, “patient” or “individual” are terms that can, in context, be used interchangeably in the present disclosure. Subjects in need of treatment include those already having an inflammatory disease as well as those in which inflammation is to be prevented, delayed or halted.

In an example, compositions of the disclosure comprise genetically unmodified MLPSCs. As used herein, the term “genetically unmodified” refers to cells that have not been modified by transfection with a nucleic acid. For the avoidance of doubt, in the context of the present disclosure a MLPSC transfected with a nucleic acid encoding a protein would be considered genetically modified.

As used herein, the term “sample” refers to an extract from a subject or cell culture in which the level of a particular marker can be measured. The “sample” includes extracts and/or derivatives and/or fractions of the sample. In an example, the sample is an extract from a subject in which CRP levels can be measured. In the present disclosure, any biological material can be used as the above-mentioned sample so long as it can be collected from the subject or cell culture and assayed to determine the level of a marker disclosed herein (e.g. level of CRP in a subject). In an example, the sample is a blood sample. For example, the blood sample can be obtained from a subject with an inflammatory disease such as GvHD.

In an example, the present disclosure encompasses selecting a population of culture expanded MLPSCs of a certain potency for use in methods of treatment disclosed herein. The term “potency” as used herein refers to the specific ability or capacity of the MLPSCs to effect a given result. In an example, the result is a therapeutic result, for example a reduction in inflammatory biomarkers as disclosed herein.

“Therapeutic efficacy” is used in the context of the present disclosure to refer to MLPSCs and compositions disclosed herein that can treat, inhibit and/or prevent disease. For example, therapeutically effective MLPSCs and compositions disclosed herein can treat, inhibit and/or prevent inflammatory disease.

“Biological activity” is used in the context of the present disclosure to define MLPSCs and compositions disclosed herein based on a particular activity. In an example, the biological activity is pro-angiogenic and/or anti-inflammatory activity. In an example, the biological activity is capacity to increase in-vitro angiogenesis. In an example, the biological activity is the increased expression of one or more angiogenic markers. In an example, biological activity is characterised reduced inflammation. In an example, biological activity is characterised reduced T-cell activation and/or proliferation.

The term “clinically proven” (used independently or to modify the term “effective”) shall mean that efficacy has been proven by a clinical trial wherein the clinical trial has met the approval standards of U.S. Food and Drug Administration, EMEA or a corresponding national regulatory agency. For example, the clinical study may be an adequately sized, randomized, double-blinded study used to clinically prove the effects of the composition. In an example, a clinically proven effective amount is an amount shown by a clinical trial to meet a specified endpoint. In an example, the end point is protection against death. Put another way, the end point increases survival. For example, 100 day survival may be increased when administering treatment according to the present disclosure.

Accordingly, the terms “clinically proven efficacy” and “clinically proven effective” can be used in the context of the present disclosure to refer to a dose, dosage regimen, treatment or method disclosed herein. Efficacy can be measured based on change in the course of the disease in response to administering a composition disclosed herein. For example, a composition of the disclosure is administered to a subject in an amount and for a time sufficient to induce an improvement, preferably a sustained improvement, in at least one indicator that reflects the severity of inflammatory disease. Various indicators that reflect the severity of the disease can be assessed for determining whether the amount and time of the treatment is sufficient. Such indicators include, for example, clinically recognized indicators of disease severity or symptoms. In an example, the degree of improvement is determined by a physician, who can make this determination based on signs, symptoms, or other test results. In an example, a clinically proven effective amount improves patient survival. In another example, a clinically proven effective amount reduces a subjects risk of mortality. In another example, a clinically proven effective amount increases 100 day survival. In an example, methods of the disclosure administer a clinically proven effective amount of a composition disclosed herein.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example disclosed herein shall be taken to apply mutatis mutandis to any other example unless specifically stated otherwise.

### Mesenchymal Lineage Precursor or Stem Cells (MLPSCs)

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “MLPSCs” includes both parent cells and their undifferentiated progeny. The term also includes mesenchymal precursor cells (MPCs), multipotent stromal cells, mesenchymal stem cells (MSCs), perivascular mesenchymal precursor cells, and their undifferentiated progeny.

MLPSCs can be autologous, allogeneic, xenogenic, syngenic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

In an example, the MLPSCs are allogeneic. In an example, the allogeneic MLPSCs are culture expanded and cryopreserved.

MLPSCs reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, MLPSCs are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for MLPSCs comprises at least about 0.1% or 0.5% or 1% or 2% or 5% or 10% or 15% or 20% or 25% or 30% or 50% or 75% MLPSCs. In this regard, the term “population of cells enriched for MLPSCs” will be taken to provide explicit support for the term “population of cells comprising X % MLPSCs”, wherein X % is a percentage as recited herein. The MLPSCs can, in some examples, form clonogenic colonies, e.g. CFU-F (fibroblasts) or a subset thereof (e.g., 50% or 60% or 70% or 70% or 90% or 95%) can have this activity.

In an example of the present disclosure, the MLPSCs are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In another example, the MLPSCs are CD29+, CD54+, CD73+, CD90+, CD102+, CD105+, CD106+, CD166+, MHC1+MSCs.

Isolated or enriched MLPSCs can be expanded in vitro by culture. Isolated or enriched MLPSCs can be cryopreserved, thawed and subsequently expanded in vitro by culture.

In one example, isolated or enriched MLPSCs are seeded at 50,000 viable cells/cm² in culture medium (serum free or serum-supplemented), for example, alpha minimum essential media (αMEM) supplemented with 5% fetal bovine serum (FBS) and glutamine, and allowed to adhere to the culture vessel overnight at 37° C., 20% 02. The culture medium is subsequently replaced and/or altered as required and the cells cultured for a further 68 to 72 hours at 37° C., 5% O₂.

As will be appreciated by those of skill in the art, cultured MLPSCs are phenotypically different to cells in vivo. For example, in one embodiment they express one or more of the following markers, CD44, NG2, DC146 and CD140b. Cultured MLPSCs are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+ (TNAP+). For example, the MPCs can be isolated from bone mononuclear cells with an anti-STRO-3 antibody.

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+ (HSP-90B), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example, MLPSCs are obtained from a single donor, or multiple donors where the donor samples or MLPSCs are subsequently pooled and then culture expanded.

MLPSCs encompassed by the present disclosure may also be cryopreserved prior to administration to a subject. In an example, MLPSCs are culture expanded and cryopreserved prior to administration to a subject.

In an example, the present disclosure encompasses MLPSCs as well as progeny thereof, soluble factors derived therefrom, and/or extracellular vesicles isolated therefrom. In another example, the present disclosure encompasses MLPSCs as well as extracellular vesicles isolated therefrom. For example, it is possible to culture expand mesenchymal precursor lineage or stem cells of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1+ cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

In an example, compositions of the disclosure comprise cells that induce new blood vessel formation in target tissue. In an example, the target tissue is the heart. In another example, the cells secrete factors that protect at risk or damaged myocardium. In an example, at risk or damaged myocardium has been subject to a lack of blood flow resulting from an ischemic event. In an example, the cells secrete factors that reduce apoptosis in cardiomyocytes.

The MLPSCs disclosed herein may be altered in such a way that upon administration, lysis of the cell is inhibited. Alteration of an antigen can induce immunological non-responsiveness or tolerance, thereby preventing the induction of the effector phases of an immune response (e.g., cytotoxic T cell generation, antibody production etc.) which are ultimately responsible for rejection of foreign cells in a normal immune response. Antigens that can be altered to achieve this goal include, for example, MHC class I antigens, MHC class II antigens, LFA-3 and ICAM-1.

The MLPSCs may also be genetically modified to express proteins of importance for the differentiation and/or maintenance of striated skeletal muscle cells. Exemplary proteins include growth factors (TGF-β, insulin-like growth factor 1 (IGF-1), FGF), myogenic factors (e.g. myoD, myogenin, myogenic factor 5 (Myf5), myogenic regulatory factor (MRF)), transcription factors (e.g. GATA-4), cytokines (e.g. cardiotropin-1), members of the neuregulin family (e.g. neuregulin 1, 2 and 3) and homeobox genes (e.g. Csx, tinman and NKx family).

### Culture Expanded MLPCs

In example, culture expanded MLPSCs of the disclosure are characterised based on therapeutic efficacy. For example, the MLPSCs may be characterised based on therapeutic efficacy in an inflammatory disease. In an example, the MLPSCs are characterised by therapeutic efficacy in heart failure. In another example, the MLPSCs are characterised by therapeutic efficacy in a T-cell mediated disease such as GvHD.

In another example, culture expanded MLPSCs are characterised by their capacity to inhibit IL-2RA expression by CD3/CD28-activated PBMCs under culture conditions. In an example, the culture expanded MLPSCs inhibit IL2-RA expression by CD3/CD28-activated PBMCs by at least 60% relative to a control. In another example, the culture expanded MLPSCs inhibit IL2-RA expression by CD3/CD28-activated PBMCs by at least 65% relative to a control. In another example, the culture expanded MLPSCs inhibit IL2-RA expression by CD3/CD28-activated PBMCs by at least 70% relative to a control. In another example the culture expanded MLPSCs inhibit IL2-RA expression by CD3/CD28-activated PBMCs by between 60 and 70% relative to a control.

“Culture expanded” MLPSCs are distinguished from freshly isolated cells in that they have been cultured in cell culture medium and passaged (i.e. sub-cultured).

In an example, freshly isolated cells are culture expanded for about 1 or 2 passages to provide an intermediate population. In an example, freshly isolated cells are culture expanded for 2 passages to provide an intermediate population. In another example, freshly isolated cells are culture expanded for about 1 to 3 passages to provide an intermediate population. In an example, freshly isolated cells are STRO-1+.

Accordingly, in an example, relevant cells are isolated and culture expanded for 2 passages to provide an intermediate MLPSC population. In certain examples, the intermediate MLPSC population is then culture expanded to provide a drug product (DP).

For example, DP compositions of the present disclosure are produced by culturing cells from an intermediate cryopreserved MLPSC population or, put another way, a cryopreserved intermediate. In an example, the intermediate cell population can be cultured for three more passages (i.e. 5 passages total) to provide a DP.

In an example, MLPSCs are culture expanded for about 4-10 passages. In an example, MLPSCs are culture expanded for at least 5, at least 6, at least 7, at least 8, at least 9, at least 10 passages. For example, MLPSCs can be culture expanded for at least 5 passages. In an example, MLPSCs can be culture expanded for at least 5-10 passages. In an example, MLPSCs can be culture expanded for at least 5-8 passages. In an example, MLPSCs can be culture expanded for at least 5-7 passages. In an example, MLPSCs can be culture expanded for more than 7 passages. In these examples, MLPSCs may be culture expanded before being cryopreserved to provide an intermediate cryopreserved MLPSC population and then subject to further culture expansion.

In an example, compositions of the disclosure comprise MLPSCs that are culture expanded from a cryopreserved intermediate. In an example, the cells culture expanded from a cryopreserved intermediate are culture expanded for at least 3, at least 5, at least 6, at least 7, at least 8, at least 9, at least 10 passages. For example, MLPSCs can be culture expanded for at least 3 passages. In an example, MLPSCs can be culture expanded for at least 3-10 passages. In an example, MLPSCs can be culture expanded for at least 3-8 passages. In an example, MLPSCs can be culture expanded for at least 3-7 passages. In an example, MLPSCs culture expanded from a cryopreserved intermediate are culture expanded in media disclosed herein (e.g. media containing newborn calf serum).

In an example, MLPSCs can be obtained from a single donor, or multiple donors where the donor samples or MLPSCs are subsequently pooled and then culture expanded as required. In an example, the culture expansion process comprises:
i. expanding by passage expansion the number of viable cells to provide a preparation of at least about 1 billion of the viable cells, wherein the passage expansion comprises establishing a primary culture of isolated MLPSCs and then serially establishing a first non-primary (P1) culture of isolated MLPSCs from the previous culture;
ii. expanding by passage expansion the P1 culture of isolated MLPSCs to a second non-primary (P2) culture of MLPSCs; and,
iii. preparing and cryopreserving an in-process intermediate MLPSC preparation obtained from the P2 culture of MLPSCs; and, optionally
iv. thawing the cryopreserved in-process intermediate MLPSC preparation and expanding by passage expansion the in-process intermediate MLPSC preparation.

In an example, the methods of the disclosure comprise selecting an intermediate population (e.g. a cryopreserved intermediate) for further culture expansion based on certain criteria such as the level of one more angiogenic markers. Selection processes are not particularly limited so long as they are able to select cell populations characterized by the relevant criteria such as level of angiogenic marker. In an example, a series of intermediate MLPSC populations are assessed for levels of angiogenic markers and those populations which express over a threshold level of the angiogenic marker as described herein are selected for further expansion.

It should be appreciated that the selection process does not require immediate culture expansion. Rather “selected” populations can be cryopreserved and culture expanded at a later stage. In an example, a fraction of the intermediate cell population is culture expanded with the remainder of the population being cryopreserved for culture expansion at a later stage.

In an example, selected cell populations are immediately culture expanded. In another example, selected cell populations are cryopreserved to allow culture expansion at a later stage.

In an example, a selected cell population is culture expanded to provide a pharmaceutical composition. In an example, the pharmaceutical composition is characterized by certain criteria such as level of angiogenic markers.

In the context of the present disclosure, the level of angiogenic marker/s can be assessed between steps iii and iv of the culture expansion process described above. For example, the level of angiogenic marker/s may be determined under culture conditions and/or from conditioned media after step iii. In an example, step iv is only performed if a desired level of angiogenic marker/s is/are observed under culture conditions and/or from conditioned media. In this example, the cell population is selected for culture expansion on the basis of the level of angiogenic marker/s under culture conditions and/or from conditioned media.

In an example, the culture expanded MLPSC population is expanded from an intermediate MLPSC population with an increased level of one or more angiogenic markers relative to a population of MLPSCs that have been culture expanded in a cell culture medium comprising 10% fetal serum.

In an example, a level of an angiogenic marker(s) disclosed herein is considered increased when it is increased by about 5%, about 10%, about 20%, about 30%, about 40%, about 50%, about 60%, or about 70% relative to a population of MLPSCs that have been culture expanded in a cell culture medium comprising 10% fetal serum. In an example, the level of angiogenic marker is increased by between about 5% and about 60%. In an example, the level of angiogenic marker is increased by between about 5% and about 40%. In an example, the level of angiogenic marker is increased by about 40%. In an example, the level of angiogenic marker is increased by at least about 5%. In an example, the level of angiogenic marker is increased by at least about 10%.

In an example, the culture expanded MLPSC population is expanded from an intermediate MLPSC population with an increased level of one or more angiogenic markers relative to a population of MLPSCs that have been culture expanded in a cell culture medium that does not comprise newborn serum. In an example, the level of angiogenic marker is considered increased when it is increased by about 5%, about 10%, about 20%, about 30%, about 40%, about 50%, about 60%, or about 70% relative to a population of MLPSCs that have been culture expanded in a cell culture medium that does not comprise newborn serum. In an example, the level of angiogenic marker is increased by between about 5% and about 60%. In an example, the level of angiogenic marker is increased by between about 5% and about 40%. In an example, the level of angiogenic marker is increased by about 40%. In an example, the level of angiogenic marker is increased by at least about 5%. In an example, the level of angiogenic marker is increased by at least about 10%.

In an example, the culture expanded MLPSC preparation has an antigen profile and an activity profile comprising:
i. less than about 0.75% CD45+ cells;
ii. at least about 95% CD 105+ cells;
iii. at least about 95% CD166+ cells.

The process of MLPSC isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although MLPSCs can generally withstand a certain amount of insult during preparation, cells are preferably manipulated by handling procedures and/or equipment that adequately performs the given step(s) while minimizing insult to the cells.

In an example, MLPSCs are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S. Pat. No. 6,251,295, which is hereby incorporated by reference.

In an example, a MLPSC composition cultured according to the present disclosure is 95% homogeneous with respect to being CD105 positive and CD166 positive and being CD45 negative. In an example, this homogeneity persists through ex vivo expansion; i.e. though multiple population doublings.

In an example, MLPSCs of the disclosure are culture expanded in 2D culture. For example, MLPSCs of the disclosure can be culture expanded in a cell factory. In certain examples, 3D culture of intermediates disclosed herein may follow using, for example, a bioreactor. In an example, MLPSCs of the disclosure are initially culture expanded in 2D culture prior to being further expanded in 3D culture. In an example, intermediate cell populations of the disclosure have not been culture expanded in 3D culture. In an example, the level of one or more angiogenic markers is assessed before subsequent culture expansion in a cell factory or 3D culture.

In an example, MLPSCs of the disclosure are culture expanded from an intermediate population. In an example, MLPSCs of the disclosure are culture expanded from the intermediate in 2D culture before seeding in 3D culture.

In the context of both intermediate populations and therapeutic compositions expanded from the same, in an example, MLPSCs of the disclosure are culture expanded in 2D culture for at least 3 days before seeding in a further culture system such as cell factory or 3D culture in a bioreactor. In an example, MLPSCs of the disclosure are culture expanded in 2D culture for at least 4 days before seeding in a further culture system. In an example, MLPSCs of the disclosure are culture expanded in 2D culture for between 3 and 5 days before seeding in a further culture system. In these examples, 2D culture can be performed in a cell factory. Various cell factory products are available commercially (e.g. Thermofisher, Sigma, Corning). In an example, the cell factory has at least 5 layers. In an example, the cell factory has at least 10 layers. In an example, the cell factory has at least 20 layers. 3D culture may be performed in various bioreactor types such as stirred tank, wave bag, and vertical wheel.

In an example, CO₂ is provided during culture expansion of MLPSCs. In an example, MLPSCs are culture expanded in less than 9% CO₂. In an example, MLPSCs are culture expanded in less than 8% CO₂. In an example, MLPSCs are culture expanded in 5% CO₂. For example, MLPSCs can be culture expanded in 5%+/−2% CO₂. In an example, the MLPSCs are culture expanded with passive priming of CO₂. For example, cell factories can be passively primed with 5% CO₂.

Priming cell factories maintains the CO₂ tension between the cell factory and incubator and stabilizes the pH level of the growth medium. Active priming involves actively passing CO₂ gas through a bacterial vent air filter into each culture vessel (e.g. cell factory) for a defined period of time (e.g. around 10 minutes). However, active priming has the potential to introduce contamination into culture as it requires an open port to provide gas. Passive priming involves placing a closed culture system into an incubator at appropriate CO₂ concentration prior to cell seeding (e.g. around 12 to 72 hours). In an example, cells of the disclosure are STRO-3+ before they are culture expanded to provide an intermediate cell population.

### Compositions

The compositions disclosed herein are useful for pre-licensing of MLPSCs, a multistep process that leads to the functional maturation of MSCs that promotes their therapeutic potency.

Accordingly provided herein is a composition for pre-licensing MLPSCs that contains a human cell population enriched for MLPSCs; and a serum, where the serum is a serum containing one or more pro-inflammatory cytokines. In other embodiments disclosed herein the composition includes (i) a human cell population enriched for MLPSCs; (ii) serum comprising one or more pro-inflammatory cytokines; and (iii) a cryopreservative. Suitable cryopreservatives include, but are not limited to one or more of: dimethylsulfoxide (DMSO), trehalose, and albumin.

In other embodiments provided herein is a cell culture medium for proliferation and pre-licensing of MLPSCs, where the cell culture medium includes a serum containing one or more pro-inflammatory cytokines.

The cell culture medium of the present disclosure can contain any components such as fatty acids or lipids, vitamins, cytokines, antioxidants, buffering agents, inorganic salts and the like.

The cell culture media used in the present disclosure contains all essential amino acids and may also contain non-essential amino acids. In general, amino acids are classified into essential amino acids (Thr, Met, Val, Leu, Ile, Phe, Trp, Lys, His) and non-essential amino acids (Gly, Ala, Ser, Cys, Gln, Asn, Asp, Tyr, Arg, Pro).

Those of skill in the art will appreciate that for optimal results, the basal medium must be appropriate for the cell line of interest with key nutrients available at adequate levels to enhance cell proliferation. For example, it may be necessary to increase the level of glucose (or other energy source) in the basal medium, or to add glucose (or other energy source) during the course of culture, if this energy source is found to be depleted and to thus limit cell proliferation.

The culture media of the present disclosure can be prepared by using a basal culture medium. In the context of the present disclosure, “basal culture medium” refers to an unsupplemented medium which is suitable for exposure to cells, for example MSC. Basal culture medium includes, for example, Eagles minimal essential (MEM) culture media, alpha modified MEM culture media, StemSpan™ and mixed culture media thereof, and is not particularly restricted providing it can be used for culturing of MLPSCs.

In some preferred embodiments of the compositions for pre-licensing disclosed herein, the composition includes one or more pro-inflammatory cytokines selected from among IL-1β, IL-6, IFN-γ, TNF-α, and IL-1 receptor antagonist (IL-1ra). In some embodiments the composition includes each of IL-1β, IL-6, IFN-Y, TNF-α, and IL-1 receptor antagonist (IL-1ra). In some embodiments the composition is substantially free of pro-inflammatory cytokines other than IL-1β, IL-6, IFN-γ, TNF-α, and IL-1ra. In some embodiments the composition contains only 1, 2, 3, 4, or 5 pro-inflammatory cytokines, where the 1 to 5 pro-inflammatory cytokines are selected from the group consisting of IL-1β, IL-6, IFN-Y, TNF-α, and IL-1ra.

In some embodiments the concentration of IL-1B in a newborn serum to be included in the compositions disclosed herein is about 2 ng/ml to about 50 ng/ml, e.g., 5 ng/ml, 10 ng/ml, 15 ng/ml, 20 ng/ml, 25 ng/ml, 30 ng/ml, 40 ng/ml, 45 ng/ml, or another concentration from about 2 ng/ml to about 50 ng/ml. In some embodiments the concentration of IL-6 in a suitable newborn serum is about 0.2 ng/ml to about 1.2 ng/ml, e.g., 0.4 ng/ml, 0.5 ng/ml, 0.6 ng/ml, 0.7 ng/ml, 0.8 ng/ml, 1.0 ng/ml, or another concentration from about 0.2 ng/ml to about 1.2 ng/ml. In some embodiments the concentration of IFN-Y in a suitable newborn serum is about 0.1 ng/ml to about 0.2 ng/ml, e.g., 0.12 ng/ml, 0.14 ng/ml, 0.16 ng/ml, 0.18 ng/ml, or another concentration from about 0.1 ng/ml to about 0.2 ng/ml. In some embodiments the concentration of IL-Ira in a suitable newborn serum is about 6 ng/ml to about 33 ng/ml, e.g., 7 ng/ml, 8 ng/ml, 9 ng/ml, 10 ng/ml, 12 ng/ml, 15 ng/ml, 20 ng/ml, 22 ng/ml, 25 ng/ml, 27 ng/ml, 30 ng/ml, or another concentration from about 6 ng/ml to about 33 ng/ml. In some embodiments the concentration of TNF-α in a suitable newborn serum is about 0.1 ng to about 0.7 ng/ml, e.g., 0.2 ng/ml, 0.3 ng/ml, 0.4 ng/ml, 0.5 ng/ml, 0.6 ng/ml, or another concentration from about 0.2 ng/ml to about 0.7 ng/ml.

In some preferred embodiments, the compositions disclosed herein are substantially free of any pro-inflammatory cytokines other than the ones present in the serum prior to its inclusion in the composition, i.e., the serum to be used is substantially the sole source of exogenous pro-inflammatory cytokines in the composition.

In some embodiments the concentration of newborn serum in the compositions described is about 2% (v/v) to about 12% (v/v), e.g., 3%, 4%, 5%, 6%, 7%, 8%, 9%, 10%, 11%, 12%, or another concentration from about 2% (v/v) to about 12% (v/v). In some preferred embodiments the concentration of newborn serum is about 5% (v/v). In other preferred embodiments the concentration of newborn serum is about 10% (v/v).

In some embodiments the compositions disclosed herein contain both fetal serum and newborn serum. In some preferred embodiments the ratio of fetal serum to newborn serum in the composition is 1:1. In some embodiments the ratio of fetal serum to newborn serum is greater than 1:1, e.g., 3:1, 2.8:1, 2.5:1, 2.2:1, 2:1, 1.8:1, 1.5:1, 1.2:1, or another ratio of fetal serum to newborn serum of about 3:1 fetal serum to newborn serum or lower. In other embodiments, the ratio of fetal serum to newborn serum is lower than 1:1, e.g., 1:3, 1:2.8, 1:2.5, 1:2.2, 1:1.8, 1:1.5, 1:1.2, or another ratio of fetal serum to newborn serum of about 1:3 fetal serum to newborn serum or higher.

In some preferred embodiments, the total concentration of serum, including fetal and newborn serum at a given ratio, in a composition is about 10% (v/v).

In some embodiments, where the composition contains a cryopreservative, e.g., DMSO, the concentration of newborn serum is from about 3% (v/v) to about 20% (v/v), e.g., 4%, 6%, 7%, 7.5%, 8%, 9%, 10%, 12%, 15%, or another concentration from about 3% (v/v) to about 15% (v/v). In some embodiments, in a cryopreservation composition, the concentration of fetal and newborn serum is at a ratio of 1:1, e.g., 5% (v/v) of each.

In some embodiments the serum containing one or more pro-inflammatory cytokines to be used in the compositions disclosed herein is a newborn serum. In some preferred embodiments the newborn serum to be used is, e.g., newborn bovine calf serum, newborn lamb serum, and newborn equine foal serum. In some preferred embodiments the newborn serum is newborn bovine calf serum. In embodiments in which a composition disclosed herein contains newborn serum, the newborn serum is from a newborn at about postnatal day 1 to about postnatal day 7, e.g., postnatal day 2, postnatal day 3, postnatal day 4, postnatal day 5, postnatal day 6. For clarity postnatal day 1, as used herein, refers to the day of birth. In some embodiments, newborn serum used in the compositions described herein can contain a mixture of newborn sera obtained from different postnatal days.

In some embodiments the newborn serum to be used is bovine, ovine, caprine, equine, or human. In some preferred embodiments the newborn serum is bovine. In some embodiments, where a composition is to contain fetal serum, the fetal serum is bovine, ovine, equine, or caprine. In some preferred embodiments, the fetal serum is bovine fetal serum.

In an example, compositions of the disclosure comprise IFN-gamma and/or TNF-alpha (e.g. serum containing IFN-gamma and TNF-alpha). For example, the level of IFN-gamma can be less than 1 ng/ml. In an example the level of IFN-gamma is less than 500 μg/ml or less than 100 μg/ml. In an example, the level of TNF-alpha can be less than 1 ng/ml. In an example, the level of TNF-alpha is less than 750 μg/ml or less than 400 μg/ml. In an example, the composition comprises IFN-gamma and TNF-alpha and, the level of both is less than 1 ng/ml. In an embodiment of this example, the IFN-gamma and TNF-alpha are provided in serum.

In an example, the composition comprises one or more pro-inflammatory cytokines which are capable of binding a receptor on the surface of MLPSCs.

In an example, the serum comprises one or more pro-inflammatory cytokines selected from the group consisting of IL-6; IL-8; IL-17A; MCP-1; MIP-1-alpha; MIP-1-beta; IP-10. For example, the serum can comprise IL-8.

In an example, the composition comprises IFN-gamma and/or TNF-alpha, and, one or more pro-inflammatory cytokines selected from the group consisting of IL-6; IL-8; IL-17A; MCP-1; MIP-1-alpha; MIP-1-beta; IP-10. In an example, the level of IFN-gamma and/or TNF-alpha is less than 1 ng/ml.

In an example, the composition may comprise serum characterised by one or more or all of the following:
i. a level of IFN-gamma greater than 10 μg/ml;
ii. a level of TNF-alpha greater than 20 μg/ml;
iii. a level of IL-6 greater than 30 μg/ml;
iv. a level of IL-8 greater than 5,000 μg/ml;
V. a level of IL-17A greater than 2 μg/ml;
vi. a level of MCP-1 greater than 30 μg/ml;
vii. a level of MIP-1-alpha greater than 5 μg/ml;
viii. a level of MIP-1-beta greater than 30 μg/ml;
ix. a level of IP-10 greater than 5,000 μg/ml.

In certain examples, the serum may be diluted from neat concentration in cell culture media. In these examples, the levels of cytokines in the serum will be reduced accordingly. For example, the serum may be provided in the cell culture media at 10% (v/v). In this example, the serum may be characterised by one or more or all of the following:
i. a level of IFN-gamma greater than 1 μg/ml;
ii. a level of TNF-alpha greater than 2 μg/ml;
iii. a level of IL-6 greater than 3 μg/ml;
iv. a level of IL-8 greater than 500 μg/ml;
V. a level of IL-17A greater than 0.2 μg/ml;
vi. a level of MCP-1 greater than 3 μg/ml;
vii. a level of MIP-1-alpha greater than 0.5 μg/ml;
viii. a level of MIP-1-beta greater than 3 μg/ml;
ix. a level of IP-10 greater than 500 μg/ml.

In an example, the serum is newborn serum, which comprises an above referenced level of cytokine(s). In an example, compositions of the disclosure comprise media described below.

In other examples, compositions of the disclosure comprises above referenced population(s) of culture expanded MLPSCs.

In some preferred embodiments the MLPSCs are human mesenchymal stem cells. In other embodiments the MLPSCs are STRO-1⁺ multipotential cells or a population of culture expanded MLPSCs that have been culture expanded from a STRO-1+ population of multipotential cells.

In preferred embodiments, the MLPSCs are maintained in an undifferentiated state.

### Media

In an embodiment the present disclosure encompasses MLPSC culture media supplemented with pro-inflammatory cytokine(s). In an example, the culture media comprises IFN-gamma and/or TNF-alpha. In an example, the media comprises IFN-gamma. For example, the level of IFN-gamma can be less than 1 ng/ml. In an example the level of IFN-gamma is less than 500 μg/ml or less than 100 μg/ml. In an example, the media comprises TNF-alpha. For example, the level of TNF-alpha can be less than 1 ng/ml. In an example, the level of TNF-alpha is less than 750 μg/ml or less than 400 μg/ml. In an example, the media comprises IFN-gamma and TNF-alpha and the level of both is less than 1 ng/ml.

In an example, the media comprises one or more pro-inflammatory cytokines which are capable of binding a receptor on the surface of MLPSCs.

In an example, the media comprises one or more pro-inflammatory cytokines selected from the group consisting of IL-6; IL-8; IL-17A; MCP-1; MIP-1-alpha; MIP-1-beta; IP-10. For example, the media can comprise IL-8.

In an example, the media comprises IFN-gamma and/or TNF-alpha, and, one or more pro-inflammatory cytokines selected from the group consisting of IL-6; IL-8; IL-17A; MCP-1; MIP-1-alpha; MIP-1-beta; IP-10. In an example, the level of IFN-gamma and/or TNF-alpha is less than 1 ng/ml.

In an example, the media is characterised by one or more or all of the following:
i. a level of IFN-gamma greater than 1 μg/ml;
ii. a level of TNF-alpha greater than 2 μg/ml;
iii. a level of IL-6 greater than 3 μg/ml;
iv. a level of IL-8 greater than 500 μg/ml;
v. a level of IL-17A greater than 0.2 μg/ml;
vi. a level of MCP-1 greater than 3 μg/ml;
vii. a level of MIP-1-alpha greater than 0.5 μg/ml;
viii. a level of MIP-1-beta greater than 3 μg/ml;
ix. a level of IP-10 greater than 500 μg/ml.

In another example, the media comprises serum which is characterised by one or more or all of the following:
i. a level of IFN-gamma greater than 10 μg/ml;
ii. a level of TNF-alpha greater than 20 μg/ml;
iii. a level of IL-6 greater than 30 μg/ml;
iv. a level of IL-8 greater than 5,000 μg/ml;
V. a level of IL-17A greater than 2 μg/ml;
vi. a level of MCP-1 greater than 30 μg/ml;
vii. a level of MIP-1-alpha greater than 50 μg/ml;
viii. a level of MIP-1-beta greater than 30 μg/ml;
ix. a level of IP-10 greater than 5,000 μg/ml.

In an example, the media comprises IL-10. In another example, the media comprises IL-36RA. In another example, the media comprises IL-10 and IL-36RA. In an example, the level of IL-10 is greater than 0.3 μg/ml. For example, the level of IL-10 may be greater than 30 μg/ml. In an example, the level of IL-10 is greater than 400 μg/ml. In an example, the level of IL-36RA is greater than 50 μg/ml.

In an example, the media is serum free.

In an example, the media is serum free and supplemented with PDGF and FGF2. In an example, the medium is serum free and is supplemented with PDGF, FGF2 and EGF. In an example, the PDGF is PDGF-BB. In an example, the serum free media is supplemented with 10 ng/ml PDGF-BB, 5 ng/ml EGF and, 1 ng/ml FGF2.

In an example, the above referenced cytokines can be provided at a concentration <1 ng/ml each. For example, the media may be characterised by one or more or all of the following, each provided at <1 ng/ml: IFN-gamma, TNF-alpha, IL-6, IL-17A, MCP-1, MIP-1-alpha, MIP-1-beta, IP-10.

### Methods

The present disclosure provides in vitro methods for pre-licensing of MLPSCs by culturing a human cell population enriched for MLPSCs (e.g., hMSCs) in a cell culture medium suitable for maintenance and proliferation of MLPSCs.

In an example, the culture medium is an above referenced composition or media. In an example, the culture medium is supplemented with a serum comprising one or more pro-inflammatory cytokines as described herein. In some preferred embodiments the culture medium to be used is supplemented with newborn serum. In some preferred embodiments the culture medium to be used is supplemented with both fetal serum and newborn serum in equal concentrations for a total serum concentration in the culture medium of about 10% (v/v). In some preferred embodiments MLPSCs are pre-licensed in cell culture medium containing 5% (v/v) newborn serum and 5% (v/v) fetal serum.

In some embodiments the methods disclosed herein include the additional step of determining or having determined the level of one or more pro-inflammatory cytokines in a serum to be included in the culture medium to be used for pre-licensing of MLPSCs. Methods for determining cytokine levels are well known in the art, e.g., ELISA.

In some embodiments the methods disclosed herein also include determining or having determined the ability of a culture medium (e.g. a newborn serum supplemented culture medium) to stimulate MLPSCs to promote angiogenesis in an in vitro assay, e.g., tube formation by human umbilical vein endothelial cells (HUVEC) and analysis of network length, network area and branch point formation. In some embodiments such an assay includes collecting MLPSC-conditioned media following its culture in a newborn serum-supplemented medium as disclosed herein and quantifying the effect of such conditioned media in the above-described angiogenesis assay or a similar assay.

In some embodiments the methods disclosed herein also include determining or having determined in the above-mentioned conditioned medium the level of one or more of Angiogenin, Angiopoietin (Ang1/ANGPT1), SDF-1α, and VEGF.

In some embodiments, where a first lot or batch of newborn serum was used in conditioned medium that promotes greater angiogenesis or release of angiogenic factors than that of conditioned medium in which second lot/batch of newborn serum was used, it is concluded that the use of the first lot of newborn serum for pre-licensing and culture expansion of MLPSCs will result in the generation of MLPSCs that have relatively greater therapeutic potency particularly for treatment of conditions where an angiogeneic or anti-inflammatory therapeutic mode of action is useful.

The methods and cell culture media of the present disclosure promote stem cell proliferation and pre-licensing while maintaining MLPSCs in an undifferentiated state. MLPSCs are considered to be undifferentiated when they have not committed to a specific differentiation lineage. As discussed above, MLPSCs display morphological characteristics that distinguish them from differentiated cells. Furthermore, undifferentiated MLPSCs express genes that may be used as markers to detect differentiation status. The polypeptide products may also be used as markers to detect differentiation status. Accordingly, one of skill in the art could readily determine whether the methods of the present disclosure maintain MLPSCs in an undifferentiated state using routine morphological, genetic and/or proteomic analysis. Methods of monitoring/confirming cell proliferation are also known in the art and, in certain examples, may be as rudimentary as periodic visual inspection of cell cultures to confirm increase in cell number. Other methods may involve the use of cell viability dyes and/or live cell imaging and counting using commercially available products.

MLPSCs disclosed herein can be culture expanded in various suitable cell culture mediums comprising newborn serum. The term “medium” or “media” as used in the context of the present disclosure, includes the components of the environment surrounding the cells. The media contributes to and/or provides the conditions suitable to allow cells to grow. Media may be solid, liquid, gaseous or a mixture of phases and materials. Media can include liquid growth media as well as liquid media that do not sustain cell growth. Exemplary gaseous media include the gaseous phase that cells growing on a petri dish or other solid or semisolid support are exposed to.

In an example, the methods of the disclosure encompass culture expansion in cell culture media which comprise one or more pro-inflammatory cytokines. In an example, the cell culture media comprises IFN-gamma and/or TNF-alpha. In an example, the cell culture media comprises IFN-gamma. For example, the level of IFN-gamma can be less than 1 ng/ml. In an example the level of IFN-gamma is less than 500 μg/ml or less than 100 μg/ml. In an example, the cell culture media comprises TNF-alpha. For example, the level of TNF-alpha can be less than 1 ng/ml. In an example, the level of TNF-alpha is less than 750 μg/ml or less than 400 μg/ml. In an example, the cell culture media comprises IFN-gamma and TNF-alpha and the level of both is less than 1 ng/ml.

In an example, the cell culture media comprises one or more pro-inflammatory cytokines which are capable of binding a receptor on the surface of MLPSCs.

In an example, the cell culture media comprises one or more pro-inflammatory cytokines selected from the group consisting of IL-6; IL-8; IL-17A; MCP-1; MIP-1-alpha; MIP-1-beta; IP-10. For example, the cell culture media can comprise IL-8.

In an example, the cell culture media comprises IFN-gamma and/or TNF-alpha, and, one or more pro-inflammatory cytokines selected from the group consisting of IL-6; IL-8; IL-17A; MCP-1; MIP-1-alpha; MIP-1-beta; IP-10. In an example, the level of IFN-gamma and/or TNF-alpha is less than 1 ng/ml.

In an example, the cell culture media is characterised by one or more or all of the following:
i. a level of IFN-gamma greater than 1 μg/ml;
ii. a level of TNF-alpha greater than 2 μg/ml;
iii. a level of IL-6 greater than 3 μg/ml;
iv. a level of IL-8 greater than 500 μg/ml;
V. a level of IL-17A greater than 0.2 μg/ml;
vi. a level of MCP-1 greater than 3 μg/ml;
vii. a level of MIP-1-alpha greater than 0.5 μg/ml;
viii. a level of MIP-1-beta greater than 3 μg/ml;
ix. a level of IP-10 greater than 500 μg/ml.

In another example, the media comprises serum characterised by one or more or all of the following:
i. a level of IFN-gamma greater than 10 μg/ml;
ii. a level of TNF-alpha greater than 20 μg/ml;
iii. a level of IL-6 greater than 30 μg/ml;
iv. a level of IL-8 greater than 5,000 μg/ml;
V. a level of IL-17A greater than 2 μg/ml;
vi. a level of MCP-1 greater than 30 μg/ml;
vii. a level of MIP-1-alpha greater than 50 μg/ml;
viii. a level of MIP-1-beta greater than 30 μg/ml;
ix. a level of IP-10 greater than 5,000 μg/ml.

In an example, the media comprises IL-10. In another example, the media comprises IL-36RA. In another example, the media comprises IL-10 and IL-36RA. In an example, the level of IL-10 is greater than 0.3 μg/ml. For example, the level of IL-10 may be greater than 30 μg/ml. In an example, the level of IL-10 is greater than 400 μg/ml. In an example, the level of IL-36RA is greater than 50 μg/ml.

In another example, methods of the disclosure encompass culture expansion in cell culture media which comprise newborn serum. Various examples of suitable serum (and levels of the same) are disclosed herein.

In an example, methods of the disclosure comprise selecting a cryopreserved intermediate population of MLPSCs for culture expansion in media disclosed herein. In an example, a cryopreserved intermedia that has been culture expanded in 10% fetal serum is selected for culture expansion according to the methods disclosed herein. In an example, a cryopreserved intermediate that has been culture expanded in newborn serum and/or pro-inflammatory cytokines disclosed herein is selected for culture expansion.

The cell culture media used for culture expansion contains all essential amino acids and may also contain non-essential amino acids. In general, amino acids are classified into essential amino acids (Thr, Met, Val, Leu, Ile, Phe, Trp, Lys, His) and non-essential amino acids (Gly, Ala, Ser, Cys, Gln, Asn, Asp, Tyr, Arg, Pro).

Those of skill in the art will appreciate that for optimal results, the basal medium must be appropriate for the cell line of interest. For example, it may be necessary to increase the level of glucose (or other energy source) in the basal medium, or to add glucose (or other energy source) during the course of culture, if this energy source is found to be depleted and to thus limit growth. In an example, dissolved oxygen (DO) levels can also be controlled.

### Serum

“Non-fetal serum” refers to serum that has been obtained postpartum. For example, the culture media can be supplemented with mammalian non-fetal serum (e.g. bovine). In an example, the culture media can be supplemented with animal non-fetal serum. In another example, the culture media can be supplemented with human non-fetal serum.

In an example, the cell culture media is supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v non-fetal serum. In an example, the cell culture media is supplemented with between about 1% v/v and about 15% v/v non-fetal serum. In an example, the cell culture media is supplemented with between about 1% v/v and about 10% v/v non-fetal serum. In an example, the cell culture media is supplemented with between about 5% v/v and about 10% v/v non-fetal serum. In an example, the cell culture media is supplemented with between about 5% v/v non-fetal serum.

In an example, the non-fetal serum comprises at least one pro-inflammatory cytokine. Methods to detect the presence of cytokines in cell culture medium and/or serum are known in the art and include, for example, enzyme-linked immunosorbent assay (ELISA). In another example, the presence of cytokines in serum are detected by measuring cytokine mRNA, for example by polymerase-chain reaction (PCR) techniques such as reverse-transcription PCR.

“Newborn serum” refers to serum that has been obtained postpartum. For example, the culture media can be supplemented with mammalian newborn serum (e.g. bovine). In an example, the culture media can be supplemented with animal newborn serum. In another example, the culture media can be supplemented with human newborn serum.

In an example, the cell culture media is supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v newborn serum. In an example, the cell culture media is supplemented with between about 1% v/v and about 15% v/v newborn serum. In an example, the cell culture media is supplemented with between about 1% v/v and about 10% v/v newborn serum. In an example, the cell culture media is supplemented with between about 5% v/v and about 10% v/v newborn serum. In an example, the cell culture media is supplemented with about 5% v/v newborn serum.

In an example, the newborn serum comprises at least one inflammatory cytokine. As used herein, the term “inflammatory cytokine” refers to a signalling molecule that promotes inflammation. In example, the one or more cytokine is selected from the group comprising IL-1β, IL-6, TNF-α, IFN-γ and/or IL-1ra.

In an example, the newborn serum comprises IFN-gamma. In another example, the newborn serum comprises TNF-alpha. In another example, the newborn serum comprises IFN-gamma and TNF-alpha. In another example, the newborn serum comprises one or more pro-inflammatory cytokines selected from the group consisting of IL-6; IL-8; IL-17A; MCP-1; MIP-1-alpha; MIP-1-beta; IP-10. For example, the newborn serum can comprise IL-8. In an example, the newborn serum comprises IFN-gamma and/or TNF-alpha and, one or more pro-inflammatory cytokines selected from the group consisting of IL-6; IL-8; IL-17A; MCP-1; MIP-1-alpha; MIP-1-beta; IP-10. In another example, the newborn serum comprises IFN-gamma and TNF-alpha and, one or more pro-inflammatory cytokines selected from the group consisting of IL-6; IL-8; IL-17A; MCP-1; MIP-1-alpha; MIP-1-beta; IP-10. In an example, the level of IFN-gamma is less than 1 ng/ml. In an example, the level of TNF-alpha is less than 1 ng/ml. In an example, the level of both IFN-gamma and TNF-alpha are less than 1 ng/ml. For example, the level of IFN-gamma may be less than 500 μg/ml or less than 100 μg/ml. In an example, the level of TNF-alpha is less than 750 μg/ml or less than 400 μg/ml.

Methods to detect the presence of cytokines in serum are known in the art and include, for example, enzyme-linked immunosorbent assay (ELISA). In another example, the presence of cytokines in serum are detected by measuring cytokine mRNA, for example by polymerase-chain reaction (PCR) techniques such as reverse-transcription PCR.

In an example, the newborn serum can be newborn calf serum (NBCS). In an example, the NBCS is obtained from newborn calves who have been fed colostrum. In an example, NBCS comprises elevated levels of at least one inflammatory cytokine relative to NBCS obtained from a calf that has not been fed colostrum. In an example, NBCS comprises elevated levels of at least one inflammatory cytokine relative to fetal serum such as FCS.

In an example, the NBCS is obtained within 4 weeks after birth of the calf. In an example, the NBCS is obtained within 21 days after birth of the calf. For example, the NBCS is obtained≤21 days after birth of the calf. In an example, the NBCS is obtained between the day of birth and 21 days after birth of the calf. In an example, the NBCS is obtained between the day of birth and 14 days after birth of the calf. In an example, the NBCS is obtained between the day of birth and 10 days after birth of the calf. In an example, the NBCS is obtained between the day of birth and 7 days after birth of the calf. In an example, the NBCS is obtained between 6 hours after birth and 72 hours after birth. In an example, the NBCS is obtained between 6 hours after birth and 48 hours after birth. In an example, the NBCS is obtained between 6 hours after birth and 24 hours after birth. In an example, the NBCS is obtained between 12 hours after birth and 24 hours after birth.

In an example, the cell culture media is supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v NBCS. In an example, the cell culture media is supplemented with between about 1% v/v and about 15% v/v NBCS. In an example, the cell culture media is supplemented with between about 5% v/v and about 10% v/v NBCS. In an example, the cell culture media is supplemented with at least about 5% v/v NBCS.

In an example, the culture medium is also supplemented with fetal serum. In an example, the fetal serum is fetal calf serum (FCS). It is envisaged that the term fetal calf serum (FCS) and fetal bovine serum (FBS) can in the context of the present disclosure be used interchangeably. In an example, cell culture medium is supplemented with less than 10% v/v FCS. In an example, cell culture medium is supplemented with about 5% v/v FCS.

In an example, the cell culture medium is fetal serum free.

In an example, the cell culture medium is FCS free.

In an example, the culture media is supplemented with a mixture of FCS and NBCS. In an example the cell culture medium is supplemented with about 5% v/v FCS and about 5% v/v NBCS (i.e. a 1:1 ratio of FCS to NBCS). In an example, the culture media can be supplemented with a mixture of FCS and NBCS so that the FCS: NBCS ratio is at least about 0.4:1, at least about 0.5:1, at least about 0.6:1, at least about 0.7:1, at least about 0.8:1, at least about 0.9:1, at least about 1:1, at least about 1.5:1, at least about 2:1. In an example, the FCS: NBCS ratio is between about 0.5:1 and about 2:1. In an example, the FCS: NBCS ratio is between about 0.8:1 and about 1.5:1. In an example, the FCS: NBCS ratio is between about 0.8:1 and about 1.2:1. In an example, the FCS: NBCS ratio is about 1:1.

In an example, the mixture of FCS and NBCS can comprise at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v of the cell culture media. In an example, the mixture of FCS and NBCS can comprise between about 1% v/v and about 15% v/v of the cell culture media. In an example, the mixture of FCS and NBCS can comprise between about 2% v/v and about 12% v/v of the cell culture media. In an example, the mixture of FCS and NBCS can comprise between about 5% v/v and about 12% v/v of the cell culture media. In an example, the mixture of FCS and NBCS can comprise between about 8% v/v and about 12% v/v of the cell culture media. In an example, the mixture of FCS and NBCS can comprise about 10% v/v of the cell culture media However, in this example, the cell culture media is supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9% v/v, but less than 10% v/v FCS. In an example, the cell culture media is supplemented with between about 1% v/v and about 9% v/v FCS. In an example, the cell culture media is supplemented with between about 3% v/v and about 8% v/v FCS. In an example, the cell culture media is supplemented with between about 3% v/v and about 6% v/v FCS. In an example, the cell culture media is supplemented with about 5% v/v FCS.

In an example, the non-fetal serum is adult serum. Accordingly, in an example, the culture media is supplemented with adult serum. In an example, the culture media is supplemented with bovine adult serum. In an example, the culture media may be supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v adult serum.

In another example, the culture media is supplemented with human adult serum, the cell culture medium can be supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9% v/v human adult serum. In an example, the cell culture medium can be supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9% v/v human AB serum. In an example, the cell culture medium is supplemented with at least about 3% human AB serum.

In another example, the culture media is supplemented with new born serum (e.g. NBCS) and adult serum. In an example, the culture media is supplemented with bovine adult serum. In an example, the culture media may be supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v adult serum.

In another example, the culture media is supplemented with human adult serum, the cell culture medium can be supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9% v/v human adult serum. In an example, the cell culture medium can be supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9% v/v human AB serum. In an example, the cell culture medium is supplemented with at least about 3% human AB serum.

### Ascorbic Acid

In an example, the cell culture media is supplemented with a short acting ascorbic acid derivative. The term “short acting” encompasses ascorbic acid derivatives that are oxidised by approximately 80-90% following 24 hours of cell culture under culture conditions of neutral pH and 37° C. In one example, the short acting L-ascorbic acid derivative is a L-ascorbic acid salt, for example L-ascorbic acid sodium salt. In an example, the cell culture media may contain at least about 0.005 g/L of a short acting ascorbic acid derivative. In another example, the cell culture media may contain at least about 0.01 g/L of a short acting ascorbic acid derivative. For example, the cell culture media may contain at least about 0.02 g/L of a short acting ascorbic acid derivative. In another example, the cell culture media may contain at least about 0.03 g/L of a short acting ascorbic acid derivative. For example, the cell culture media may contain at least about 0.04 g/L of a short acting ascorbic acid derivative. In another example, the cell culture media may contain at least about 0.05 g/L of a short acting ascorbic acid derivative. In another example, the cell culture media may contain at least about 0.06 g/L of a short acting ascorbic acid derivative.

In another example, the cell culture media contains a short acting ascorbic acid derivative but does not contain a substantial amount of a long acting ascorbic acid derivative. For example, the cell culture media may contain a short acting ascorbic acid derivative but not more than 0.04 g/L of a long acting ascorbic acid derivative. In another example, the cell culture media may contain a short acting ascorbic acid derivative but not more than 0.03 g/L of a long acting ascorbic acid derivative. In another example, the cell culture media may contain a short acting ascorbic acid derivative but not more than 0.02 g/L of a long acting ascorbic acid derivative. In another example, the cell culture media may contain a short acting ascorbic acid derivative but not more than 0.01 g/L of a long acting ascorbic acid derivative. In another example, the cell culture media may contain a short acting ascorbic acid derivative but not more than 0.005 g/L of a long acting ascorbic acid derivative. In another example, the cell culture media may contain a short acting ascorbic acid derivative but not a long acting ascorbic acid derivative. In another example, the cell culture media contains L-ascorbate sodium salt but does not contain a substantial amount of L-ascorbic acid-2-phosphate.

### Other Additives

In an example, the cell culture medium contains human derived additives. For example, human serum and human platelet cell lysate can be added to the cell culture media. In other examples, additional factors can be added to the cell culture medium. For example, the cell culture media can be supplemented with one or more stimulatory factors selected from the group consisting of, platelet derived growth factor (PDGF), fibroblast growth factor 2 (FGF2), epidermal growth factor (EGF), epidermal growth factor (EGF), 1α,25-dihydroxyvitamin D3 (1,25D), tumor necrosis factor α (TNF-α), interleukin-1β (IL-1β) and stromal derived factor 1α (SDF-1α). In another embodiment, cells may also be cultured in the presence of at least one cytokine in an amount adequate to support growth of the cells. In another embodiment, cells can be cultured in the presence of heparin or a derivative thereof.

In the above examples, basal medium such as Alpha MEM or StemSpan™ can be supplemented with the referenced quantity of serum and, in certain examples, other additives. Further examples of suitable culture mediums for culturing stem cells can be found, for example, in WO2016139340.

### Angiogenic Markers

According to the present disclosure, in certain embodiments, MLPSCs cultured according to the methods disclosed herein express increased levels of one or more angiogenic markers. In an example, methods of the disclosure relate to selection of culture expanded MLPSCs for use in treatments such as treatment of inflammation. Such methods comprise determining the level(s) of a marker(s) disclosed herein and, selecting for use in treatment MLPSCs that have increased levels of one or more of the marker(s).

In certain examples, methods of the disclosure involve measuring the level of pro-angiogenic factors, such as VEGF, angiogenin and/or SDF-1α, expressed by MLPSCs under culture conditions. In an example, MLPSCs can be culture expanded in culture media according to the methods disclosed herein. Conditioned media from cultured MLPSCs is then isolated (i.e. a sample is obtained from the cell culture) and the amount of expressed angiogenic marker contained therein is measured. The level of angiogenic markers in MLPSC-conditioned media can be measured by standard protein detection methods and/or gene expression methods known in the art. In an example, the level of angiogenic marker is measured by enzyme-linked immunosorbent assay (ELISA). For example, conditioned media of MLPSCs is obtained and then contacted with anti-VEGF antibody, anti-SDF-1α antibody, and/or an anti-angiogenin antibody. Extent of antibody binding is used to quantify the level of angiogenic marker in the conditioned media (e.g. ng/L). In this example, the level of angiogenic marker in the conditioned media is a measure of the level of angiogenic marker expressed or secreted by MLPSCs.

In an example, the level of angiogenic marker is measured by a Western blot. In an example, the level of angiogenic marker is measured by a Luminex assay. In an example, the level of angiogenic marker is measured by reverse transcription RT-PCR.

In an example, MLPSCs are selected for use in treatment if they express elevated levels of vascular endothelial growth factor (VEGF). In an example, level of VEGF is greater than about 3 ng/ml. In an example, the level of VEGF is greater than between about 3 ng/ml and 4 ng/ml. In an example, level of VEGF is greater than about 3.1 ng/ml. In an example, level of VEGF is greater than about 3.2 ng/ml. In an example, level of VEGF is greater than about 3.3 ng/ml. In an example, level of VEGF is greater than about 3.4 ng/ml. In an example, level of VEGF is greater than about 3.5 ng/ml. In an example, the level of VEGF is between about 3 ng/ml and 4 ng/ml. In an example, the level of VEGF is between about 3.2 and 3.6 ng/ml. In an example, the level of VEGF is about 3.45 ng/ml.

In an example, MLPSCs are selected for use in treatment if they have increased levels of VEGF relative to a population of MLPSCs that have been culture expanded in a cell culture medium comprising 10% fetal serum. In an example, the level of VEGF is increased by about 5%, about 10%, about 20%, about 30%, about 40%, about 50%, about 60%, or about 70% relative to a population of MLPSCs that have been culture expanded in a cell culture medium comprising 10% fetal serum. In an example, the level of VEGF is increased by between about 5% and about 60%. In an example, the level of VEGF is increased by between about 5% and about 40%. In an example, the level of VEGF is increased by about 40%. In an example, the level of VEGF is increased by at least about 5%. In an example, the level of VEGF is increased by at least about 10%.

In an example, MLPSCs are selected for use in treatment if they express elevated levels of angiogenin. In an example, the level of angiogenin is greater than about 1000 μg/ml. In an example, the level of angiogenin is greater than about 1100 μg/ml. In an example, the level of angiogenin is between about 1000 μg/ml and 1200 μg/ml. In an example, the level of angiogenin is between about 1100 μg/ml and 1150 μg/ml. In an example, the level of angiogenin is about 1114 μg/ml or higher.

In an example, MLPSCs are selected for use in treatment if they have increased levels of angiogenin relative to a population of MLPSCs that have been culture expanded in a cell culture medium comprising 10% fetal serum. In an example, the level of angiogenin is increased by about 5%, about 10%, about 20%, about 30%, about 40%, about 50%, about 60%, or about 70% relative to a population of MLPSCs that have been culture expanded in a cell culture medium comprising 10% fetal serum. In an example, the level of angiogenin is increased by between about 5% and about 60%. In an example, the level of angiogenin is increased by between about 5% and about 40%. In an example, the level of angiogenin is increased by about 40%. In an example, the level of angiogenin is increased by at least about 5%. In an example, the level of angiogenin is increased by at least about 10%.

In an example, MLPSCs are selected for use in treatment if they express elevated levels of stromal derived factor 1α (SDF-1α). In an example, the level of SDF-1α is greater than about 3000 ng/ml. In an example, the level of SDF-1α is greater than about 3100 ng/ml. In an example, the level of SDF-1α is greater than about 3200 ng/ml. In an example, the level of SDF-1α is greater than about 3300 ng/ml. In an example, the level of SDF-1α is greater than about 3400 ng/ml. In an example, the level of SDF-1α is greater than about 3500 ng/ml. In an example, the level of SDF-1a is between about 3000 ng/ml and 3500 ng/ml. In an example, the level of SDF-1α is between about 3000 ng/ml and 3400 ng/ml. In an example, the level of SDF-1α is between about 3000 ng/ml and 3300 ng/ml. In an example, the level of SDF-1α is between about 3100 ng/ml and 3400 ng/ml. In an example, the level of SDF-1α is between about 3100 ng/ml and 3300 ng/ml.

In an example, MLPSCs are selected for use in treatment if they have increased levels of SDF-1a relative to a population of MLPSCs that have been culture expanded in a cell culture medium comprising 10% fetal serum. In an example, the level of SDF-1α is increased by about 5%, about 10%, about 20%, about 30%, about 40%, about 50%, about 60%, or about 70% relative to a population of MLPSCs that have been culture expanded in a cell culture medium comprising 10% fetal serum. In an example, the level of SDF-1α is increased by between about 5% and about 60%. In an example, the level of SDF-1α is increased by between about 5% and about 40%. In an example, the level of SDF-1α is increased by about 40%. In an example, the level of SDF-1α is increased by at least about 5%. In an example, the level of SDF-1α is increased by at least about 10%.

In another example, the angiogenic marker is increased angiogenesis. In this example, increased angiogenesis is measured by an in-vitro angiogenesis assay, for example, a quantitative live-cell imaging assay. Briefly, an endothelial cell line (e.g. human umbilical vein endothelial cells (HUVECs), human dermal fibroblasts, human saphenous vein endothelial cells (HSaVECs), human coronary artery endothelial cells (HCAECs), human aortic endothelial cells (HAECs), brain microvascular endothelial cells (BMEC), or any combination thereof) are fluorescently labelled and seeded into a culture plates. The endothelial cells are then simultaneously incubated in the presence or absence of MLPSC-conditioned media and imaged using a live-cell imaging system.

In this example, angiogenesis can be measured by various network morphometric parameters identified and computed by image analysis software as composite of various elements described in Table 1 (Lam et al. Biomaterials 290. (2022) 121826). In an example, the live-cell imaging system is the IncuCyte® Live-Cell Analysis System. Live-cell imaging systems enable the fluorescent identification of cells and visualization of angiogenesis over time by time-lapse image acquisition. Images can be analysed using computer-based image analysis tools. In an example, the image analysis tool is the IncuCyte® Angiogenesis Analysis Software Module. The IncuCyte® Angiogenesis Analysis Software Module measures angiogenic outputs including endothelial network length, endothelial network area and endothelial branch point formation. The skilled person will appreciate that other image analysis applications can used, for example Image J, CellProfiler. Other examples of live imaging in-vitro angiogenesis assays are disclosed, for example, in Lam et al. Biomaterials 290. (2022). 121826.

TABLE 1
Network morphometric parameters

| Element | Definition |
|---|---|
| Node | Pixel having at least three neighbours |
| Junction | Group of nodes forming a bifurcation |
| Master Junction | Junctions linked with at least three master segments |
| Extremity | Pixel having only one neighbour |
| Segment | Binary line linked with two junctions |
| Master Segment | Binary lines linked with two junctions that are not |
| | exclusive to one branch |
| Branch | Binary line linked with one junction and one extremity |
| Isolated Element | Binary line connected to two extremities |
| Mesh | Area enclosed by segments and corresponding junctions |
| Number of Extremities | Number of Extremities in image |
| Number of Nodes | Number of Nodes in image |
| Number of Junctions | Number of Junctions in image |
| Number of Master | Number of Master Junctions in Image |
| Junctions | |
| Number of Master | Number of Master Segments in Image |
| Segments | |
| Total Master Segment | Sum of all measured Segments in image |
| Length | |
| Number of Meshes | Number of meshes in image |
| Total Mesh Area | Sum of mesh areas in image |
| Number of Pieces | Number of segments, isolated elements, and branches in |
| | image |
| Number of Segments | Number of segments in image |
| Number of Branches | Number of branches in image |
| Number of Isolated | Number of Isolated Segments in image |
| Segments | |
| Total Length | Sum of ‘Branch’, ‘Segment’, and ‘Isolated Segment’ |
| | lengths of all vessels in image |
| Total Branching Length | Sum of all ‘Branch’ and ‘Segment’ lengths in image |
| Total Segments Length | Sum of all ‘Segment’ lengths in image |
| Total Branches Length | Sum of all ‘Branch’ lengths in image |
| Total Isolated Branches | Sum of all ‘Isolated Element’ lengths in image |
| Length | |
| Branching Interval | Mean distance separating two branches in image |
| Mesh Index | Mean distance separating two master junctions in image |
| Mean Mesh Size | Mean of ‘Mesh’ area in image (unit - pixel) |

In an example, angiogenesis is measured by the level of endothelial network formation, endothelial network length, and/or endothelial branch length. In an example, the level of endothelial network formation, endothelial network length, and/or endothelial branch length is calculated by the IncuCyte® Angiogenesis Analysis Software Module.

In another example, the endothelial network formation, endothelial network length, and/or endothelial branch length can be calculated as a composite of one or more of number of nodes, number of junctions, number of segments, number of meshes, mean mesh size, total mesh area, number of extremities, total branches length, and/or number of branches. Further examples of how endothelial network formation endothelial network length, and/or endothelial branch length can be calculated are described, for example, in Lam et al. Biomaterials 290. (2022).

In an example, MLPSCs are selected for use in treatment if they increase the level of one or more of endothelial network formation, endothelial network length, and/or endothelial branch length. In an example, MLPSCSs are selected when endothelial network formation is greater than about 0.1 mm²/mm². In an example, the endothelial network formation is between about 0.1 mm²/mm² and 0.2 mm²/mm². In an example, the endothelial network formation is about 0.12 mm²/mm². In an example, the endothelial network length is greater than about 4 mm²/mm². In an example, the endothelial network length is between about 4 mm²/mm² and about 6 mm²/mm². In an example, the endothelial network length is about 5 mm²/mm². In an example, the endothelial branch length is greater than about 12 1/mm². In an example, the endothelial branch length is between about 12 1/mm² and about 17 1/mm². In an example, the endothelial branch length is about 15 1/mm².

In an example, MLPSCs are selected for use in treatment if they increase the level of one or more of endothelial network formation, endothelial network length, and/or endothelial branch length relative to a population of MLPSCs that have been culture expanded in a cell culture medium comprising 10% fetal serum. In an example, the level of endothelial network formation is increased by about 5%, about 10%, about 20%, about 30%, about 40%, about 50%, about 60%, or about 70% relative to a population of MLPSCs that have been culture expanded in a cell culture medium comprising 10% fetal serum. In an example, the level of endothelial network formation is increased by between about 5% and about 60%. In an example, the level of endothelial network formation is increased by between about 5% and about 40%. In an example, the level of endothelial network formation is increased by about 40%. In an example, the level of endothelial network formation is increased by at least about 5%. In an example, the level of endothelial network formation is increased by at least about 10%.

In an example, MLPSCs are selected when the level of endothelial network length is increased by about 5%, about 10%, about 20%, about 30%, about 40%, about 50%, about 60%, or about 70% relative to a population of MLPSCs that have been culture expanded in a cell culture medium comprising 10% fetal serum. In an example, the level of endothelial network length is increased by between about 5% and about 60%. In an example, the level of endothelial network length is increased by between about 5% and about 40%. In an example, the level of endothelial network length is increased by about 40%. I an example, the level of endothelial network length is increased by at least about 5%. In an example, the level of endothelial network length is increased by at least about 10%.

In an example, MLPSCs are selected when the level of endothelial branch length is increased by about 5%, about 10%, about 20%, about 30%, about 40%, about 50%, about 60%, or about 70% relative to a population of MLPSCs that have been culture expanded in a cell culture medium comprising 10% fetal serum. In an example, the level of endothelial branch length is increased by between about 5% and about 60%. In an example, the level of endothelial branch length is increased by between about 5% and about 40%. In an example, the level of endothelial branch length is increased by about 40%. In an example, the level of endothelial branch length is increased by at least about 5%. In an example, the level of endothelial branch length is increased by at least about 10%.

### Compositions

MLPSCs disclosed herein can be culture expanded from a cryopreserved intermediate to produce a preparation containing at least one therapeutic dose.

In an example, compositions of the disclosure comprise around 150 million cells.

In one example, compositions of the disclose comprise a pharmaceutically acceptable carrier and/or excipient. The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. Such a media composition does not induce any adverse effects in a subject to whom it is administered. Exemplary carriers and excipients do not adversely affect the viability of a cell and/or the ability of a cell to treat or prevent disease.

In one example, the carrier or excipient provides a buffering activity to maintain the cells and/or soluble factors at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

Compositions of the disclosure may be cryopreserved. Cryopreservation of MLPSCs can be carried out using slow-rate cooling methods or ‘fast’ freezing protocols known in the art. Preferably, the method of cryopreservation maintains similar phenotypes, cell surface markers and growth rates of cryopreserved cells in comparison with unfrozen cells.

The cryopreserved composition may comprise a cryopreservation solution. The pH of the cryopreservation solution is typically 6.5 to 8, preferably 7.4.

The cyropreservation solution may comprise a sterile, non-pyrogenic isotonic solution such as, for example, PlasmaLyte ATM. 100 mL of PlasmaLyte ATM contains 526 mg of sodium chloride, USP (NaCl); 502 mg of sodium gluconate (C6H11Na07); 368 mg of sodium acetate trihydrate, USP (C2H3NaO2.3H20); 37 mg of potassium chloride, USP (KCl); and 30 mg of magnesium chloride, USP (MgCl2·6H2O). It contains no antimicrobial agents. The pH is adjusted with sodium hydroxide. The pH is 7.4 (6.5 to 8.0).

The cryopreservation solution may comprise Profreeze™ The cryopreservation solution may additionally or alternatively comprise culture medium, for example, αMEM.

To facilitate freezing, a cryoprotectant such as, for example, dimethylsulfoxide (DMSO), is usually added to the cryopreservation solution. Ideally, the cryoprotectant should be nontoxic for cells and patients, nonantigenic, chemically inert, provide high survival rate after thawing and allow transplantation without washing. However, the most commonly used cryoprotector, DMSO, shows some cytotoxicity. Hydroxylethyl starch (HES) may be used as a substitute or in combination with DMSO to reduce cytotoxicity of the cryopreservation solution.

The cryopreservation solution may comprise one or more of DMSO, hydroxyethyl starch, human serum components and other protein bulking agents. In one example, the cryopreserved solution comprises Plasma-Lyte A (70%), DMSO (10%), HSA (25%) solution, the HSA solution comprising 5% HSA and 15% buffer.

In an example, the cryopreservation solution may further comprise one or more of methycellulose, polyvinyl pyrrolidone (PVP) and trehalose.

The cryopreserved composition may be thawed and administered directly to the subject or added to another solution, for example, comprising hyaluronic acid. Alternatively, the cryopreserved composition may be thawed and the MLPSCs resuspended in an alternate carrier prior to administration.

The compositions described herein may be administered alone or as admixtures with other cells. The cells of different types may be admixed with a composition of the disclosure immediately or shortly prior to administration, or they may be co-cultured together for a period of time prior to administration.

In one example, the composition comprises an effective amount or a therapeutically or prophylactically effective amount of MLPSCs and/or progeny thereof and/or soluble factor derived therefrom. For example, the composition comprises about 1×10⁵ stem cells to about 1×10⁹ stem cells or about 1.25×10³ stem cells to about 1.25×10⁷ stem cells/kg (80 kg subject). The exact amount of cells to be administered is dependent upon a variety of factors, including the age, weight, and sex of the subject, and the extent and severity of the disorder being treated.

Despite the number of cells provided in the composition, in an example, 50×10⁶ to 200×10⁷ cells are administered. In other examples, 60×10⁶ to 200×10⁶ cells or 75×10⁶ to 150×10⁶ cells are administered. In an example, 75×10⁶ cells are administered. In another example, 150×10⁶ cells are administered.

In an example, the composition comprises greater than 5.00×10⁶ viable cells/mL. In another example, the composition comprises greater than 5.50×10⁶ viable cells/mL. In another example, the composition comprises greater than 6.00×10⁶ viable cells/mL. In another example, the composition comprises greater than 6.50×10⁶ viable cells/mL. In another example, the composition comprises greater than 6.68×10⁶ viable cells/mL.

In an example, the MLPSCs comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of the cell population of the composition.

In an example, the composition may optionally be packaged in a suitable container with written instructions for a desired purpose.

Compositions of the disclosure may be administered systemically, such as, for example, by intravenous administration. In an example, compositions are administered transendocardially.

In an example, compositions of the disclosure comprise a “clinically proven effective” amount of MLPSCs. In an example, compositions of the disclosure comprise a “clinically proven effective” amount of MSCs. In an example, compositions of the disclosure comprise a “clinically proven effective” amount of MPCs.

In an example, the “clinically proven effective” amount of MLPSCs is administered as a total dose. The term “total dose” is used in the context of the present disclosure to refer to the total number of cells received by the subject treated according to the present disclosure. In an example, the total dose consists of one administration of cells. In another example, the total dose consists of two administrations of cells. In another example, the total dose consists of three administrations of cells. In another example, the total dose consists of four or more administrations of cells. For example, the total dose can consist of two to four administrations of cells.

### Methods of Treatment

Methods of the present disclosure relate to treating inflammation in a subject by administering to the subject a composition comprising a population MLPSCs as disclosed herein. In an example, methods of treatment disclosed herein reduce T-cell activation and/or proliferation. In another example, methods of treatment disclosed herein improve patient survival.

As used herein, the terms, “inflammation” and “inflammatory disease” should be taken to encompass any disease with an inflammatory component, for example autoimmune diseases, pain, infectious diseases, cardiovascular diseases, cancer, inflammatory lung diseases, inflammatory bowel diseases, trauma, and/or neurodegenerative diseases. Examples of inflammatory diseases include, but are not limited to, graft versus host disease (GvHD) pruritus, skin inflammation, psoriasis, multiple sclerosis, rheumatoid arthritis, osteoarthritis, systemic lupus erythematosus, Hashimoto's thyroidis, myasthenia gravis, diabetes type I or II, diabetic nephropathy, asthma, inflammatory lung injury, inflammatory liver injury, inflammatory glomerular injury, atopic dermatitis, allergic contact dermatitis, irritant contact dermatitis, seborrhoeic dermatitis, Sjoegren's syndrome, keratoconjunctivitis, uveitis, inflammatory bowel disease, Crohn's disease, ulcerative colitis, an inflammatory disease of the joints, skin, or muscle, acute or chronic idiopathic inflammatory arthritis, myositis, a demyelinating disease, chronic obstructive pulmonary disease (COPD), interstitial lung disease, interstitial nephritis and chronic active hepatitis.

In an example, the inflammatory disease is refractory to primary therapy. In an example, the inflammatory disease is refractory to steroid immunosuppressant therapy. For example, the inflammatory disease is refractory to systemic steroids. In an example, the steroid is a corticosteroid. In another example, the steroid is a glucocorticoid. In another example, the steroid is prednisone. In an example, the inflammatory disease is refractory to a biologic therapy. For example, the inflammatory disease is refractory to etanercept, infliximab, ruxolitinib, anti-thymocyte globulin, mycophenolate, alemtuzumab, basiliximab, or tocilizumab. In an example, the inflammatory disease is refractory to extracorporeal photopheresis. In an example, the inflammatory disease is refractory to steroid immunosuppressant therapy. In an example, the inflammatory disease is refractory to steroid therapy and a second line of therapy. In an example, the second line of therapy is ruxolitinib.

In an example, the inflammatory disease is an autoimmune disease. In an example, the inflammatory disease is rheumatoid arthritis.

In an example, the inflammatory disease is a cardiovascular disease. In an example, the inflammatory disease is caused by a pulmonary embolism or a thrombosis such as a venous thrombosis or an arterial thrombosis. The term “thrombosis” is used herein to refer to the formation of a thrombus or blood clot. In an example, the thrombosis is “arterial thrombosis” where the blood clot develops in an artery. Such blood clots are particularly dangerous to a subject as they can obstruct blood flow to major organs such as the heart or brain. The term “pulmonary embolism” is used herein to refer to a blockage of an artery in the lungs by a substance that has moved from elsewhere in the body through the bloodstream.

In an example, the inflammatory disease is associated with aberrant T-cell proliferation. In an example, the inflammatory disease is mediated by T-cell activation.

### Graft Versus Host Disease

In an example, the inflammatory disease is GvHD. GvHD is an immunological disorder that is the major factor that limits the success and availability of allogeneic bone marrow or stem cell transplantation. In an example, the inflammatory disease is acute GvHD. Acute GvHD usually manifests within 100 days following bone marrow or stem cell transplantation. In an example, the inflammatory disease is chronic GvHD. Chronic GvHD generally manifests later than acute GvHD (>100 days post transplantation) and has some features of autoimmune diseases. It may develop either de novo, following resolution of aGvHD or as an extension of aGvHD. Chronic GvHD can cause multiple, often debilitating symptoms, including widespread skin rashes, painful mouth ulcers, shortness of breath, and limb and joint pain.

In an example, the inflammatory disease is severe GvHD. Severe GvHD can be acute or chronic.

GvHD severity can also be graded by patterns of organ involvement and clinical performance status. Multi-organ involvement includes skin rash, liver involvement, and/or gastrointestinal (GI) involvement. In an example, the subject has GvHD with multi-organ involvement. Severe GvHD can be assessed, for example, via the IBMTR Severity Index for acute GvHD (Rowlings et al., (1997) British Journal of Haematology; 97, 855-864), Glucksberg clinical stage and grade of acute GvHD (Glucksberg et al., (1974); Transplantation; 18, 295-304), and/or MAGIC Algorithm Probability (MAP), (Major-Monfried et al., (2018) Blood; 131 (25): 2846-2855; Hartwell et al., (2017) JCI Insight.; 2 (3): e89798).

In an example, severe GvHD is graded according to the Glucksberg scale (Glucksberg et al, 1974; Thomas et al, 1975) (Table 2). For example, the subject can have Grade II GvHD or Grade III/IV GvHD according to the Glucksberg scale. In one example, the subject has Grade II GvHD. In another example, the subject has Grade III/IV GvHD.

In another example, severe GvHD is graded according to IBMTR Severity Index (Table 3) (Rowlings et al., 1997). In one example, the subject has Grade B, Grade C, or Grade D GvHD according to the IBMTR severity scale.

In another example, the subject has Minnesota high risk GvHD. Minnesota high risk acute GvHD is defined as either skin stage 4; lower gastrointestinal (GI) stage 3-4 or liver stage 3-4; or skin stage 3+ and either lower GI 2-4 or liver stage 2-4 GvHD (MacMillan et al., 2015). In each of these examples, the subject can also have a high MAP score. For example, the subject can have a MAP score ≥0.29.

TABLE 2
Glucksberg clinical stage and grade
of acute GvHD (Rowlings et al., 1997)

| Stage | Skin | Liver | Intestinal tract |
|---|---|---|---|
| 1 | Maculopapular | Bilirubin 34-50 | >500 ml |
| | rash <25% of body | μmol/l | diarrhoea/d |
| | surface | | |
| 2 | Maculopapular rash | Bilirubin 51-102 | >1000 ml |
| | 25-50% of body | μmol/l | diarrhoea/d |
| | surface | | |
| 3 | Generalised | Bilirubin 103-225 | >1500 ml |
| | erythroderma | μmol/l | diarrhoea/d |
| 4 | Generalised | Bilirubin >225 | Severe abdominal |
| | erythroderma with | μmol/l | pain, with or |
| | bullous formation | | without ileus |
| | and desquamation | | |

| Grade | Degree of organ involvement |
|---|---|
| I | Stage 1-2 skin rash: no gut involvement: no liver involvement: |
| | no decrease in clinical performance |
| II | Stage 1-3 skin rash: stage 1 gut involvement or stage 1 liver |
| | involvement (or both): mild decrease in clinical performance |
| III | Stage 2-3 skin rash: stage 2-3 gut involvement or stage 2-4 liver |
| | involvement (or both): marked decrease in clinical performance |
| IV | Similar to Grade III with stage-4 organ involvement and extreme |
| | decrease in clinical performance |

TABLE 3
Criteria for IBMTR Severity Index for
acute GvHD (Rowlings et al., 1997)

| | Skin | | | Liver | | | Gastrointestinal | |
|---|---|---|---|---|---|---|---|---|
| | involvement | | | involvement | | | invovlement | |
| | | Extent | | | Total | | | Volume of |
| In- | Stage | of | | Stage | bilirubin | | Stage | diarrhoea |
| dex* | (max) | rash | | (max) | (μmol/l) | | (max) | (ml/d) |
| A | 1 | <25% | | 0 | <34 | | 0 | <500 |
| B | 2 | 25-50% | or | 1-2 | 34-102 | or | 1-2 | 550-1500 |
| C | 3 | >50% | or | 3 | 103-255 | or | 3 | >1500 |
| D | 4 | Bullae | or | 4 | >255 | or | 4 | Severe |
| | | | | | | | | pain and |
| | | | | | | | | ileus |

*Assign Index based on maximum in an individual organ system

In an example, severe GvHD presents in the stomach and/or gut of the subject as damage to these organs. In this example, various sites of inflammation can occur along the stomach and/or gut and, in certain examples, it may be preferable to administer treatment directly to one or more of these sites of inflammation. In an example, severe GvHD is accompanied by inflammatory bowel disease (IBD). For example, the GvHD may be accompanied by Crohn's disease. Accordingly, in an example, the inflammatory disease is inflammatory bowel disease. In an example, the inflammatory disease is Crohn's disease.

In an example, treatment of GvHD induces a partial response after treatment is initiated. In an example, the partial response is induced 28 days after treatment is initiated. In an example, a partial response is characterized by one or more or all of:
Reduction in Skin % BSA score of at least one point;
Reduction in mouth score of at least one point;
Reduction in eye score of at least one point;
Reduction in skin features score of at least one point;
Reduction in gastrointestinal tract score of at least one point;
Reduction in liver score of at least one point;
Reduction in lung symptom score of at least one point;
Reduction in lung FEV1 score of at least one point;
Reduction in joints and fascia score of at least one point;
Reduction in genital tract score of at least one point.

In another example, a partial response is characterized by one or more or all of:
Reduction in Skin % BSA score of at least one point;
Reduction in mouth score of at least one point;
Reduction in eye score of at least one point.

In an example, treatment of GvHD induces a complete response after treatment is initiated. In an example, the complete response is induced 28 days after treatment is initiated.

### Hyperinflammation

In an example, the inflammatory disease is hyperinflammation. The term “hyperinflammation” is used in the context of the present disclosure refer to severe and ongoing inflammatory process in body. For example, hyperinflammation can refer to severe and ongoing inflammatory process in airway and/or lungs, kidney or liver. In this way, hyperinflammation can affect multiple organs in the body and their vasculature. In an example, hyperinflammation is associated with a cytokine storm or cytokine release syndrome (CRS). In an example, the cytokine storm or CRS involves significant release of inflammatory cytokines such as IL-6.

In an example, the hyperinflammation leads to secondary (or acquired) hemophagocytic lymphohistiocytosis (sHLH). Accordingly, in an example, the methods of the present disclosure encompass treatment of hemophagocytic lymphohistiocytosis (HLH).

In another example, hyperinflammation is associated with elevated CRP, PCT, IL-6 and/or ferritin. For example, ferritin may be greater than 2000 mg/dl. In another example, ferritin is greater than 2500 mg/dl. In an example, hyperinflammation is associated with bacterial infection. In an example, subjects treated according to the present disclosure have elevated circulating CRP levels. For example, subjects treated according to the present disclosure can have circulating CRP levels greater than 100 mg/dl. In another example, treated subjects have circulating CRP levels greater than 120 mg/dl. In another example, treated subjects have circulating CRP levels greater than 150 mg/dl. In another example, treated subjects have circulating CRP levels between 90 mg/dl and 300 mg/dl.

In other examples, hyperinflammation is associated with elevated triglycerides or decreased fibrinogen. For example, subjects treated according to the present disclosure can have triglyceride levels >1.5 mmol/L. In another example, treated subjects have triglyceride levels >2, >3, >4 mmol/L. In another example, treated subjects have triglyceride levels between 1.5 and 5 mmol/L. In another example, treated subjects have fibrinogen levels less than or equal to 2.5 g/L. In another example, treated subjects have fibrinogen levels less than 2.5 g/L.

In an example, the hyperinflammation leads to multi-system inflammatory syndrome (MIS). For example, hyperinflammation can lead to MIS in children (MIS-C).

In an example, hyperinflammation is caused by a viral infection. For example, the hyperinflammation can be caused by a rhinovirus, an influenza virus, a respiratory syncytial virus (RSV) or a coronavirus. In an example, the hyperinflammation can be caused by a coronavirus. For example, the coronavirus can be coronavirus (SARS-COV), Middle East Respiratory Syndrome coronavirus (MERS-COV) or COVID-19. In an example, the hyperinflammation is caused by Epstein-Barr virus (EBV) or herpes simplex virus (HSV).

### Multi-System Inflammatory Syndrome

In an example, the inflammatory disease is multi-system inflammatory syndrome (MIS). In an example, the subject is a child with MIS. For example, a subject with MIS can be between 1 month and 18 years old. In an example, the subject has acute heart failure. Heart failure (HF) is a clinical syndrome generally characterised by a constellation of symptoms (dyspnoea, orthopnoea, lower limb swelling) and signs (elevated jugular venous pressure, pulmonary congestion). Acute heart failure is broadly defined as a rapid onset of new or worsening signs and symptoms of heart failure. In an example, the subject has depressed left ventricular ejection fraction. For example, the subjects LVEF may be less than 45%. In an example, the LVEF is less than 40%. In another example, the LVEF is less than 30%.

In an example, a subject with MIS meets the following criteria: One or more of the following:
Hypotension or shock (cardiogenic or vasogenic)
Features of severe cardiac illness including but not limited to myocarditis, pericarditis, or valvulitis, significantly elevated troponin/proBNP, or coronary artery abnormalities.
Other severe end-organ involvement, including but not limited to neurological or renal disease (excluding severe respiratory disease alone);
or,
Two or more of the following:
Maculopapular rash
Bilateral non-purulent conjunctivitis
Mucocutaneous inflammatory signs (mouth, hands, or feet)
Acute gastrointestinal symptoms (diarrhea, vomiting, or abdominal pain).

In an example, subjects meeting the above referenced MIS criteria have a fever (temperature greater than or equal to 38 degrees Celsius). In another example, a subject with MIS presents with elevated inflammatory biomarkers. For example, the subject can present with one or more of neutrophilia, lymphopenia, thrombocytopenia, hypoalbuminemia, elevated CRP, erythrocyte sedimentation rate (ESR), fibrinogen, D-dimer, ferritin, lactic acid dehydrogenase (LDH), interleukin 6 (IL-6), elevated procalcitonin. In an example, the subject has two or more of neutrophilia, lymphopenia, thrombocytopenia, hypoalbuminemia, elevated CRP, ESR, fibrinogen, D-dimer, ferritin, LDH, IL-6, elevated procalcitonin.

In an example, the subject has one or more of myocarditis, pericarditis, or valvulitis. In an example, the subject has viral induced myocarditis, pericarditis, or valvulitis. For example, the subject can have viral myocarditis.

In an example, the MIS is secondary to infection with SARS-COV, MERS-COV or COVID-19.

### Inflammatory Lung Diseases

In an example, the inflammatory disease is an inflammatory lung disease. The term “inflammatory lung disease” is used in the context of the present disclosure refer to diseases which result from ongoing inflammatory process in the airway and/or lungs. For example, COPD is inflammatory lung disease where both the airways and lung tissue are affected. This can manifest as a combination of chronic obstructive bronchitis and emphysema, where the former is the result of chronic inflammation of the bronchial tubes and the latter is due to breakdown of the alveoli. Other examples of inflammatory lung diseases include Acute Respiratory Distress Syndrome (ARDS), Idiopathic Pulmonary Fibrosis (IPF), Pulmonary Arterial Hypertension (PAH), asthma, cystic fibrosis, pneumonia and interstitial lung diseases.

In an example, the inflammatory lung disease is caused by a viral infection. For example, the inflammatory lung disease can be caused by a rhinovirus, an influenza virus, a respiratory syncytial virus (RSV) or a coronavirus. In an example, the inflammatory lung disease can be caused by a coronavirus. For example, the coronavirus can be coronavirus (SARS-COV), Middle East Respiratory Syndrome coronavirus (MERS-CoV) or COVID-19. In an example, the inflammatory lunch disease is characterized by a combination of the above referenced indications. For example, the inflammatory lung disease may comprise COPD and ARDS.

In an example, the inflammatory lung disease is acute respiratory distress syndrome (ARDS). The term “acute respiratory distress syndrome (ARDS)” is a type of respiratory failure characterized by widespread inflammation in the lungs, poor oxygenation and non-compliant or “stiff” lungs. The disorder is generally associated with capillary endothelial injury and diffuse alveolar damage.

Severity of ARDS can be diagnosed depending on the PaO2/FiO2 ratio. For example, severity of ARDS can be diagnosed as follows: (Mild: 26.6 kPa<PaO2/FiO2≤39.9 kPa; Moderate: 13.3 kPa<PaO2/FiO2≤26.6 kPa; Severe: PaO2/FiO2≤13.3 kPa). In an example, severity of ARDS can be diagnosed according to the Berlin definition as summarised in Table 4 below:

| | |
|---|---|
| Timing | Within 1 week of a known clinical insult or new/worsening respiratory |
| | symptoms. |
| Chest | Bilateral opacities not fully explained by effusions. Lobar/lung collapse or |
| imaging | nodules. |
| Origin of | Respiratory failure not fully explained by cardiac failure or fluid overload. |
| oedema | Needs objective assessment (e.g. echocardiography) to exclude hydrostatic |
| | oedema if no risk factor present. |
| Oxygenation | Mild-26.6 kPa < PaO2/FiO2 ≤ 39.9 kPa with PEEP or CPAP ≥ 5 cm H₂0 |
| | Moderate-13.3 kPa < PaO2/FiO2 ≤ 26.6 kPa with PEEP or CPAP ≥ 5 cm |
| | H₂0. |
| | Severe- PaO2/FiO2 ≤ 13.3 kPa with PEEP ≥ 5 cm H₂O. |

In another example, severity of ARDS can be diagnosed as follows: mild (PaO2/FiO2 200 to 300 mmHg); moderate (PaO2/FiO2 100 to 200 mmHg); severe (PaO2/FiO2 less than 100 mmHg).

In an example, the ARDS is mild ARDS. In another example, the ARDS is moderate ARDS. In another example, the ARDS is severe ARDS. In another example, the ARDS is moderate or severe ARDS. In an example, subjects with moderate to severe ARDS have a circulating CRP level >4 mg/L. In another example, the ARDS is moderate, severe or very severe ARDS. In an example, the subject with ARDS is greater than or equal to 65 years old. In an example, the subject with ARDS is less than 65 years old.

In an example, ARDS is caused by a viral infection. For example, the ARDS can be caused by a rhinovirus, an influenza virus, a respiratory syncytial virus (RSV) or a coronavirus. In an example, the ARDS can be caused by a coronavirus. For example, the coronavirus can be coronavirus (SARS-COV), Middle East Respiratory Syndrome coronavirus (MERS-COV) or COVID-19. In an example, the ARDS is caused by Epstein-Barr virus (EBV) or herpes simplex virus (HSV).

In another example, the ARDS is caused by a thrombosis. In another example, the ARDS is caused by an embolism. In an example, ARDS is caused by a pulmonary embolism.

In another example, the ARDS is secondary to hemophagocytic lymphohistiocytosis (HLH). HLH is a life-threatening disease characterized by lymphocyte and macrophage hyperinflammation. HLH can be triggered by viral infections such as EBV, CMV, HHV. Accordingly, in an example, the HLH is secondary or acquired HLH. For example, the HLH can be secondary to viral infection and lead to the development of ARDS in a subject.

In an example, the inflammatory disease is COPD. In an example, the COPD is mild COPD, moderate COPD, severe COPD, or very severe COPD. In an example, the severity of COPD is determined based on Global Initiative for Obstructive Lung Disease (GOLD) criteria for COPD (see for example, Rabe et al., (2007) Respir Crit Care Med., 176:532-555). In this example, subjects with COPD can have an FEV1/FVC ratio of <0.70. In an example, in subjects with an FEV1/FVC ratio of <0.70 the following is used to diagnose the severity of COPD:
GOLD 1-mild: FEV1≥80% predicted;
GOLD 2-moderate: 50%≤FEV1<80% predicted;
GOLD 3-severe: 30%≤FEV1<50% predicted;
GOLD 4-very severe: FEV1<30% predicted.

### Viral Infections

In an example, the inflammatory disease is caused by a viral infection such as a rhinovirus, an influenza virus, a respiratory syncytial virus (RSV) or a coronavirus. In an example, the viral infection is caused by a coronavirus. In an example, the coronavirus is Severe Acute Respiratory Syndrome coronavirus (SARS-COV), Middle East Respiratory Syndrome coronavirus (MERS-COV) or COVID-19.

### Diabetes

In an example, the inflammatory disease is diabetes. Diabetes Mellitus (DM) is a diagnostic term for a group of disorders characterized by abnormal carbohydrate (e.g., glucose) homeostasis or metabolism resulting in elevated blood sugar. These disorders comprise several interrelated metabolic, vascular, and neuropathic components. Various components of DM are caused by endocrine and/or exocrine functions of the pancreas. For example, the metabolic component, generally characterized by hyperglycemia, comprises alterations in carbohydrate, fat and protein metabolism caused by absent or markedly reduced secretion of hormones, particularly insulin (i.e., endocrine function) and/or ineffective insulin action. At an exocrine level, the pancreas produces various enzymes that are involved in digestion of food. For example, the pancreas produces amylase and in DM may secrete insufficient levels of this enzyme to digest carbohydrate leading to exocrine pancreatic insufficiency, malnutrition and weight loss.

The vascular component of DM comprises abnormalities in the blood vessels leading to cardiovascular, retinal and renal complications. Abnormalities in the peripheral and autonomic nervous systems are also components of DM. Vascular complications can also give rise to impaired wound healing, necrosis and gangrene. In an example, the inflammatory disease is an associated condition or symptom of diabetes selected from the group consisting of abnormal wound healing, kidney failure, blindness, neuropathy, nephropathy, retinopathy, inflammation, impotence or nonalcoholic steatohepatitis (NASH). In an example, the inflammatory disease is diabetic nephropathy.

There are several types of diabetes, including Type I (also referred to as insulin-dependent diabetes mellitus or IDDM) and Type II (also referred to as non-insulin-dependent diabetes mellitus or NIDDM), gestational diabetes and pre-diabetes (or impaired glucose metabolism). In an example, the inflammatory disease is type I diabetes. In an example, the inflammatory disease is type II diabetes.

### Pain

In an example, the inflammatory disease is pain. Pain can be a chronic condition associated with inflammation, pain and has a significant effect on disability and quality of life.

In an example, the pain is chronic pain. In an example, the pain is lower back pain. For example, the lower back pain is associated with a degenerated disc. In an example, the lower back pain is associated with an intervertebral disc (IVD). The IVD provides flexibility and mechanical stability to the spinal column during axial compression, flexion and extension. The IVD is composed of several specialised connective tissues: (i) the hyaline cartilage of the cartilaginous end plates (CEPs) which cover the surface of the vertebral bones (bodies) which are positioned above and below the disc; (ii) the fibrocartilagenous annulus fibrosus (AF) which encapsulates the nucleus pulposus (NP); and (iii) the central gelatinous nucleus pulposus (NP). The IVD can undergo degeneration due to ageing or trauma. Common IVD lesions that result in chronic pain include i) the rim lesion, a transverse defect close to the attachment of the AF to the bone of the vertebral body rim; (ii) the concentric (circumferential) tear, where the annular lamellae separate from each other; and (iii) the radiating tear which results from the propagation of clefts initiating within the NP. These lesions can trigger a local inflammatory response which can exacerbate the pain experienced by an individual.

In an example, the pain is associated with a disc that can have a disc height that is not substantially reduced compared to that of an adjacent healthy disc in the subject. In another example, the lower back pain is non-radicular in origin. In another example, the pain is associated with one or more of an intervertebral disc herniation up to a 3 mm protrusion; nerve ingrowth into an intervertebral disc; or, inflammation in an intervertebral disc. In an example, the nerve ingrowth or inflammation is in the intervertebral disc space, or the nucleus pulposus, or the annulus fibrosis of the intervertebral disc.

In an example, the pain is axial pain. The term “axial pain” is used in the context of the present disclosure to refer to localised pain, confined to a certain spot or region (e.g. neck pain or leg pain). In an example, the axial pain is due to nerve root compression defined by MRI.

### Selecting a Subject for Treatment

In an example, the methods of the present disclosure relate to methods of selecting a subject with an inflammatory disease such as a T-cell mediated inflammatory disease for treatment with stem cell compositions according to the disclosure.

In another example, the method comprises the steps of: i) selecting a subject having an inflammatory disease disclosed herein, and ii) administering to the subject a composition comprising MLPSCs, wherein the MLPSCs have been culture expanded in a cell culture medium comprising non-fetal serum. In an example, the subject has persistent inflammation as characterised by circulating CRP levels. In an example, the subject has GvHD.

### EXAMPLES

### Example 1: Serum Analysis

Mesenchymal precursor lineage or stem cell populations were culture expanded in either 5% FCS/5% NBCS (serum A) or 10% fetal bovine serum (serum B). These MLPSCs were used in examples 4-6.

Cytokine levels in 5% FCS/5% NBCS (serum A) and 10% fetal bovine serum (serum B) were assessed. To provide an external control, cytokine levels were also assessed in FBS from a different supplier (serum C). In each instance, cytokine levels were assessed in neat serum.

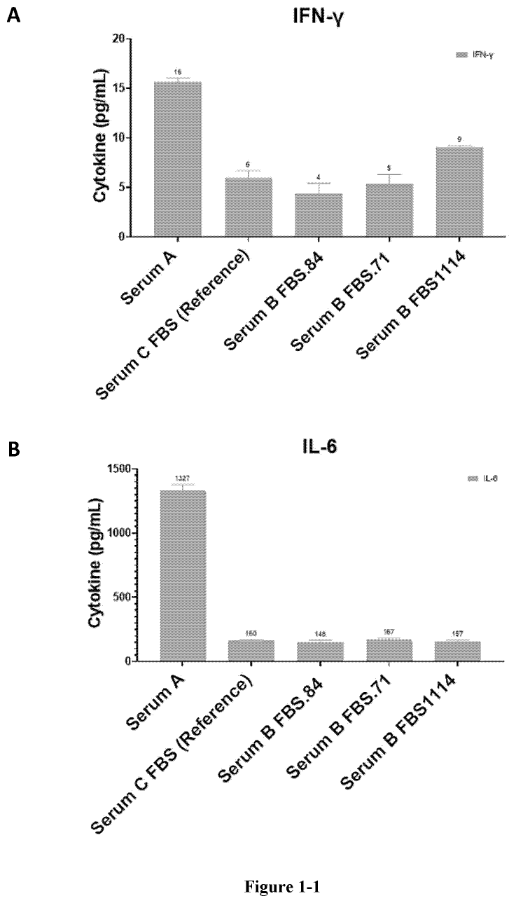

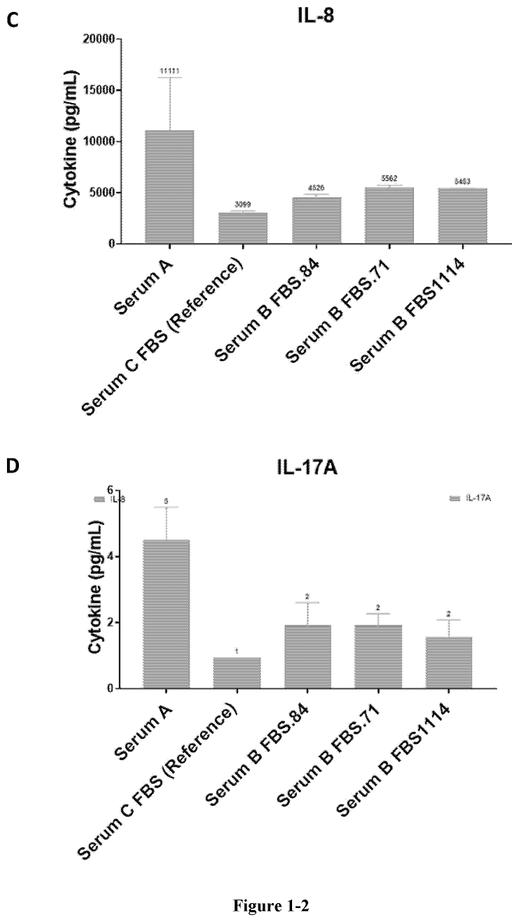

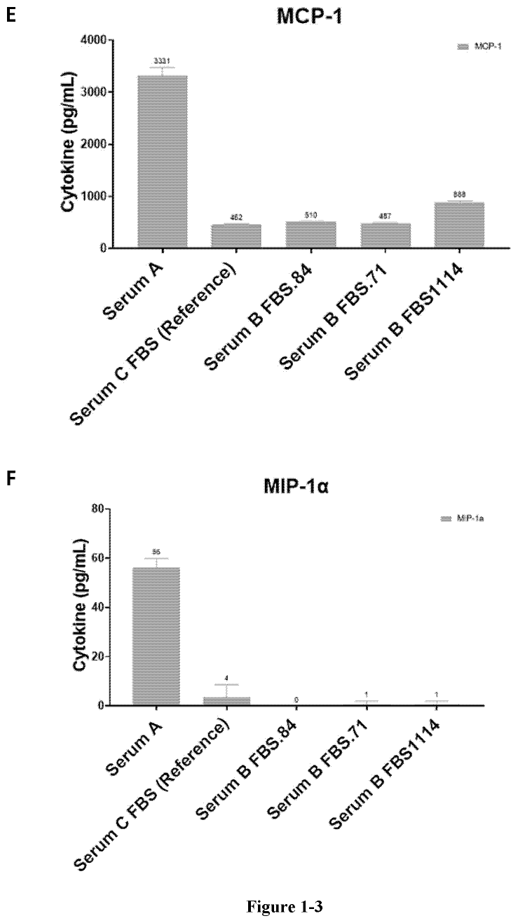

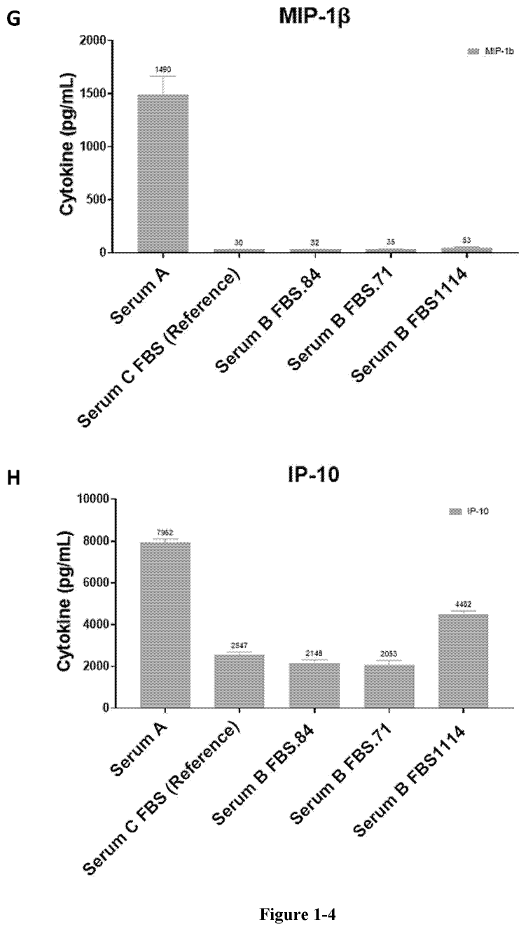

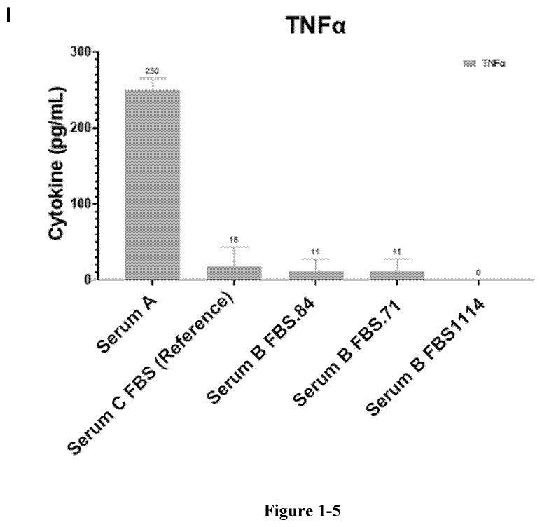

Surprisingly, pro-inflammatory cytokine levels were higher in serum preparations containing newborn calf serum (FIG. 1). Notably, there was an increase in pro-inflammatory cytokines known to bind receptors expressed on the surface of MLPSCs, such as interferon gamma (IFNγ), tumor necrosis factor alpha (TNFα) and, interleukins. For example, the following was observed in serum preparations containing newborn calf serum relative to fetal bovine serums:
At least a 2x increase in IFNγ;
At least an 13x increase in TNFα;
At least an 8x increase in IL-6;
At least an 2x increase in IL-8;
At least an 2x increase in IL-17A.

### Example 2: MLPSC Compositions Derived Using Culture Media Comprising Fetal Serum

The Alpha modification of Eagle's minimum essential media (MEM) with Earle's balanced salts, commonly referred to as Eagle's Alpha MEM, contains non-essential amino acids, sodium pyruvate, and additional vitamins. These modifications were first described for use in growing hybrid mouse and hamster cells (Stanners et al. 1971).

Eagle's Alpha MEM media suitable for culturing primary stem cells can be obtained from a variety of sources, including Life Technologies and Sigma.

A detailed method of establishing primary stem cell cultures, including the required growth factors used in the Exemplified processes is described in Gronthos and Simmons 1995.

Eagle's Alpha MEM media supplemented with 10% fetal calf serum (serum B), L-ascorbate-2-phosphate (100 μM), dexamethasone (10-7 M) and/or inorganic phosphate (3 mM) was used for culturing MLPSCs.

### Example 3: Licensing Culture Media (Non-Fetal Serum Culture Media)

For the MLPSC culture media comprising newborn serum, the serum component of the Eagle's Alpha MEM culture media described in Example 2 was modified by supplementing with 5% (v/v) newborn serum (Differences in the fetal serum media and newborn serum media are shown in Table 5). The newborn serum used was newborn calf serum (NBCS; serum A). NBCS was 100% bovine serum obtained from animals meeting the standard fetal bovine serum specifications but under the age of 20 days after birth.

NBCS was obtained from a commercial supplier, where it is marketed as an FCS substitute that is highly similar to FCS, to be used interchangeably, and expected to perform the same on cell lines.

TABLE 5
Summary of the differences between fetal serum
culture media and licensing culture media

| | |
|---|---|
| Fetal serum | |
| culture media | Licensing culture media |

Media (Change applicable to Thaw Feed, Passage)

| | |
|---|---|
| Alpha MEM | 50 mg/L short acting ascorbic acid derivative |
| 10% v/v FCS | (Sodium L-ascorbate) replaces long acting |
| | ascorbic acid derivative (L-ascorbic acid-2- |
| | phosphate) |
| | 5% v/v FCS |
| | 5% v/v newborn serum |

Cryopreservation Formulation (50% Alpha-
MEM/42.5% ProFreeze/7.5% DMSO)

| | |
|---|---|
| Alpha MEM | 50 mg/L short acting ascorbic acid derivative |
| 10% v/v FCS | (Sodium L-ascorbate) replaces long acting |
| | ascorbic acid derivative (L-ascorbic acid-2- |
| | phosphate) |
| | 5% v/v FCS |
| | 5% v/v newborn serum |

### Example 4: Culture Expansion of MLPSCs in Media Supplemented with Newborn Serum Enhances Angiogenesis

With a view to characterising the novel MLPSC populations derived by culture expansion in media supplemented with newborn serum and/or pro-inflammatory cytokines (and to potentially identify a mechanism for the observed increase in therapeutic efficacy described in Example 5), we assessed the angiogenic potential of MPCs cultured under different conditions.

Cell culture: MPCs were cultured with either 5% NBCS/5% FCS (serum A) or 10% FCS (serum B) to generate MPC-conditioned media. To control for donor variation, MPCs were obtained from the same donor and then cultured under different conditions.

In some experiments, MPCs belonging to same donor but cultured during a different manufacturing expansion are indicated by separate “lot” numbers.

Conditioned media was obtained by separating the cells from the culture media supernatant. Briefly, cryopreserved MPCs were thawed and seeded at 50,000/cm² in alpha MEM and either 10% FBS or 5% NBCS/5% FCS. Conditioned media (CM) was collected after incubating the cells for 72 h at 37° C. 5% CO₂. VEGF, SDF-1 and angiogenin levels in CM were measured using Luminex (R&D Systems). The CM was concentrated using a 3 k protein concentration filtration column (Amicon® Ultra-15) and reconstituted back to 1× or 0.25× in Assay medium.

Angiogenesis potency assay: In-vitro angiogenesis was measured using a kinetic, quantitative 96-well co-culture angiogenesis model. Lentivirus-transduced human umbilical vein endothelial cells (HUVEC) expressing CytoLight Green (a GFP variant) cultured with normal human dermal fibroblasts (NHDF) were seeded into 96-well plates and simultaneously incubated and imaged using the IncuCyte® Live-Cell Analysis System. This system enables the fluorescent identification of HUVEC (CytoLight Green+) cells and allows visualization of tube formation over time by time-lapse image acquisition. The acquired images are analysed using an integrated angiogenesis algorithm to measure network length, network area and branch point formation to provide a quantitation of the stage and extent of angiogenesis throughout the assay.

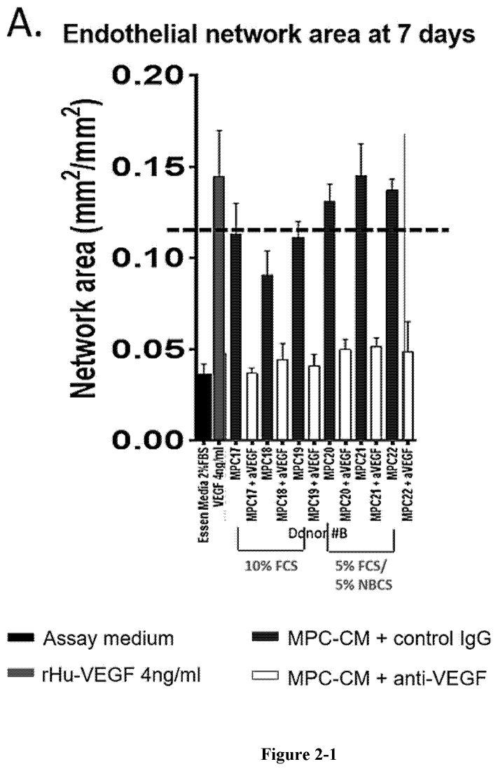

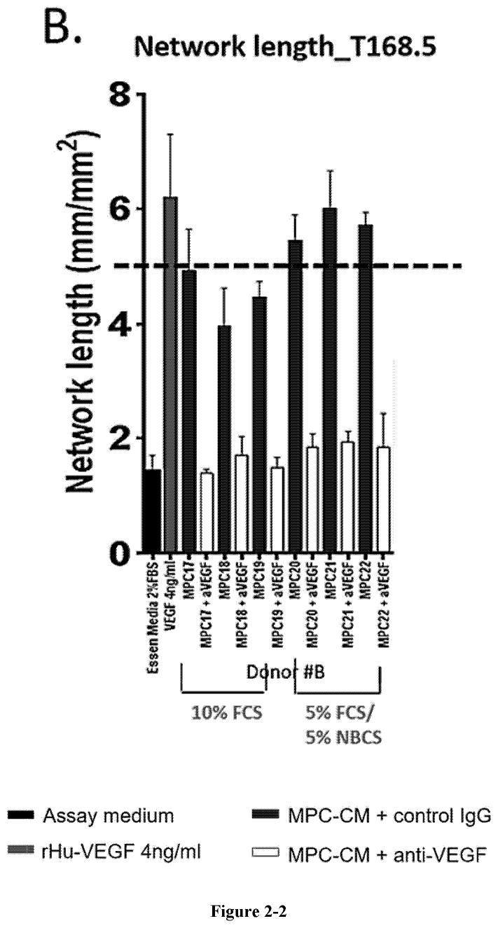

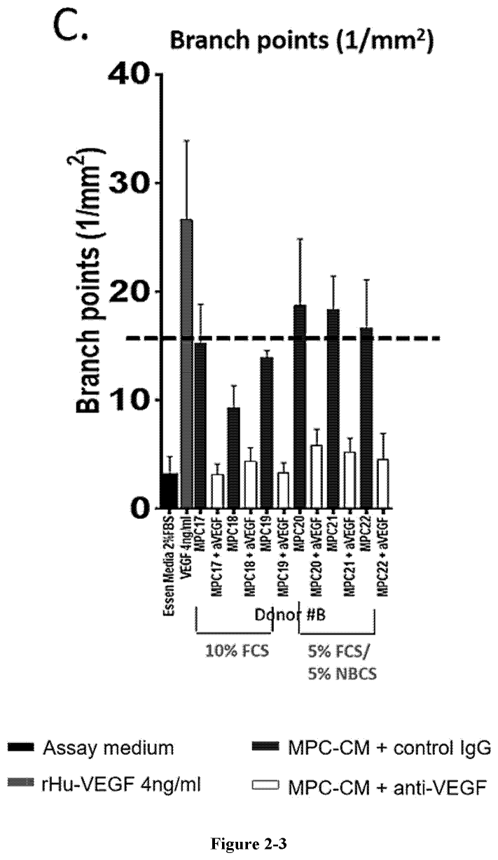

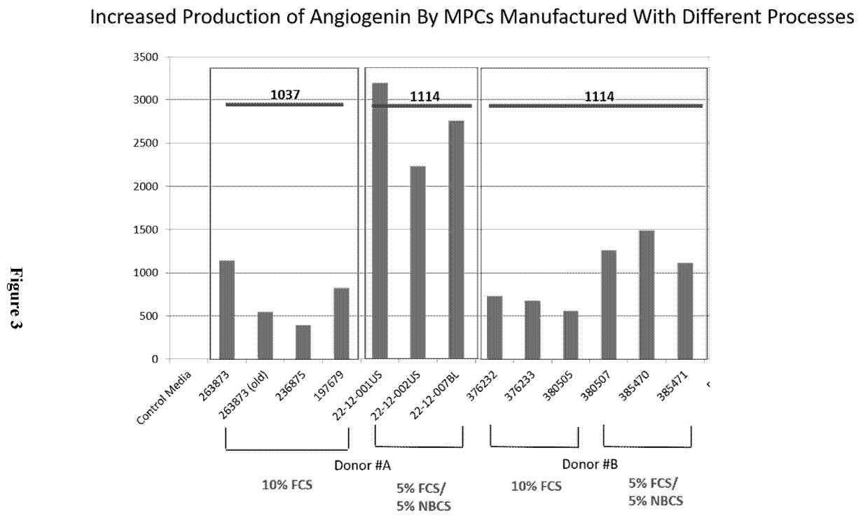

Results: Conditioned media from MPCs cultured in media supplemented with newborn calf serum was found to increase angiogenesis. As shown in FIG. 2, conditioned media from MPCs cultured in 5% NBCS/5% FCS increased network area (FIG. 2A), network length (FIG. 2B) and branch points (FIG. 2C) in the co-culture angiogenesis model. Furthermore, conditioned media from MPCs cultured in 5% NBCS/5% FCS comprises higher levels of VEGF compared to conditioned media from cells cultured in 10% FCS (FIG. 2A). Angiogenin levels were also increased in conditioned media from MPCs cultured in 5% NBCS/5% FCS when compared to 10% FCS (FIG. 3). FIG. 4 shows further analysis of the levels of angiogenic factors SDF-1α, VEGF and Ang1 (ANGPT1) present in MPCs cultured in either 10% FCS (“serum B Media”) or 5% FCS/5% NBCS (“serum A Media”). These data show that both VEGF and SDF-1α are elevated in newborn serum media-cultured MPCs.

In view of the data provided in Example 1, these data indicate that culture expansion of MLPSCs in media supplemented with newborn serum and/or proinflammatory cytokines provides novel cell populations with enhanced angiogenic potential. This enhanced potential may be, if required, characterised in various ways (for example, to define the novel cell populations identified by the present inventors) including, for example:
Capacity for conditioned media derived from the MLPSCs to increase network area, network length and/or branch points when contacted with HUVECs;
Angiogenin, VEGF and/or SDF-1 levels in conditioned media.

### Example 5: MLPSCs Cultured in Media Supplemented with Newborn Serum Improve Therapeutic Outcomes in the Context of Persistent Inflammation

NYHA Class II/III high-risk heart failure with reduced ejection fraction (HFrEF) is a clinical model of persistent inflammation. HFrEF patients are characterized by cardiac and systemic inflammation, as determined by the presence of elevated inflammatory biomarkers. MPCs cultured under different serum conditions were administered to HFrEF patients in the clinical study described below.

In HFrEF patients, cardiac macrophages produce high levels of pro-inflammatory cytokines (IL-6, IL-1, TNF-alpha) which cause endothelial dysfunction and cardiomyocyte apoptosis. Plasma C-Reactive Protein (CRP) levels, as determined by a high sensitivity CRP (hsCRP) assay, reflect hepatic production of acute phase reactants in response to the high levels of pro-inflammatory cytokines (IL-6, IL-1 and TNF-alpha) produced by cardiac macrophages. Accordingly, plasma hsCRP levels (<2 mg/L vs>2 mg/L) are representative systemic measurements reflective of low or high intra-cardiac inflammation. In the following study, HFrEF patients were categorized as having persistent inflammation if their plasma hsCRP levels were >2 mg/L.

Study details: Eligible NYHA Class II/III patients were enrolled in the Double-blind, Randomized, Sham-procedure-controlled, Parallel-Group Efficacy and Safety Study of Allogeneic Mesenchymal Precursor Cells (Rexlemestrocel-L) in Chronic Heart Failure Due to LV Systolic Dysfunction (Ischemic or Nonischemic) (DREAM HF-1) trial. HFrEF patients were administered: (1) MPCs cultured in 10% fetal serum (n=37), (2) MPCs cultured in the presence of newborn calf serum (5% FCS/5% NBCS; n=153) or, (3) a sham control (i.e., no MPCs; n=241). As evidenced by the serum analysis described in Example 1, cells cultured in media supplemented with newborn serum were effectively cultured in media comprising increased levels of pro-inflammatory cytokines. Cells were administered in a single transendocardial injection. LV systolic function in HFrEF was measured by echocardiogram (ECHO) parameters including left ventricular ejection fraction (LVEF; %), left ventricular end-systolic volume (LVESV; mL), and left ventricular end-diastolic volume (LVEDV; mL) at baseline and 12 months post treatment. Plasma CRP levels were measured to determine baseline levels of inflammation.

Results: MPCs cultured in the presence of newborn calf serum (5% FCS/5% NBCS) were found to improve left ventricular (LV) systolic function in HFrEF patients at 12 months. In particular, newborn serum-cultured MPCs significantly increased LVEF and decreased LVESV compared to sham controls (p=0.0398 and 0.0426, respectively) (FIG. 5).

HFrEF patients were then characterised according to plasma hsCRP levels of either <2 mg/L (normal baseline systemic inflammation) or >2 mg/L (elevated baseline systemic inflammation). Importantly, when HFrEF patients were differentiated according to baseline systemic inflammation status (CRP>2), the effect of treatment with MPCs cultured in the presence of newborn calf serum (5% FCS/5% NBCS) LV systolic functional recovery induced was more pronounced. In contrast, MPCs cultured in 10% FBS did not induce a significant effect (FIG. 6). Newborn serum-cultured MPCs significantly increased LVEF % by an average (LS mean) of 2.46 and decreased L VESV by an average of 8.99 mL compared to sham controls (p=0.0033 and 0.0264, respectively). In contrast, MPCs cultured in 10% fetal serum or 5%/FCS/5% NBCS showed improvements to LV systolic function in HFrEF patients without elevated baseline inflammation (HFrEF patients with CRP<2) (FIG. 7).

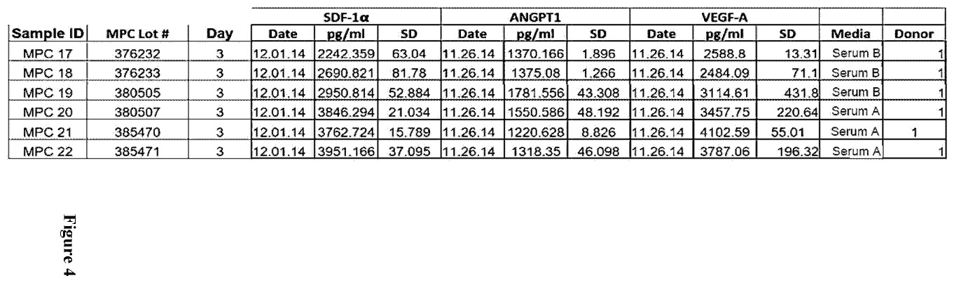

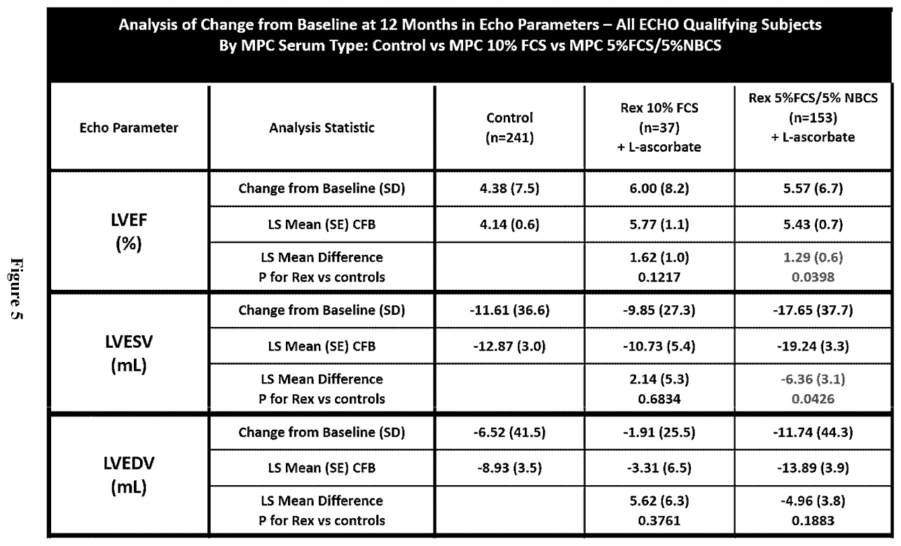

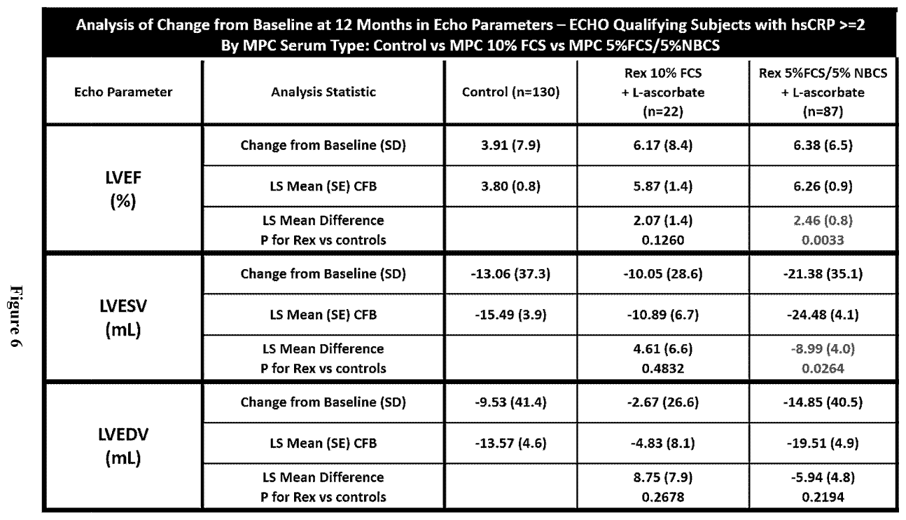

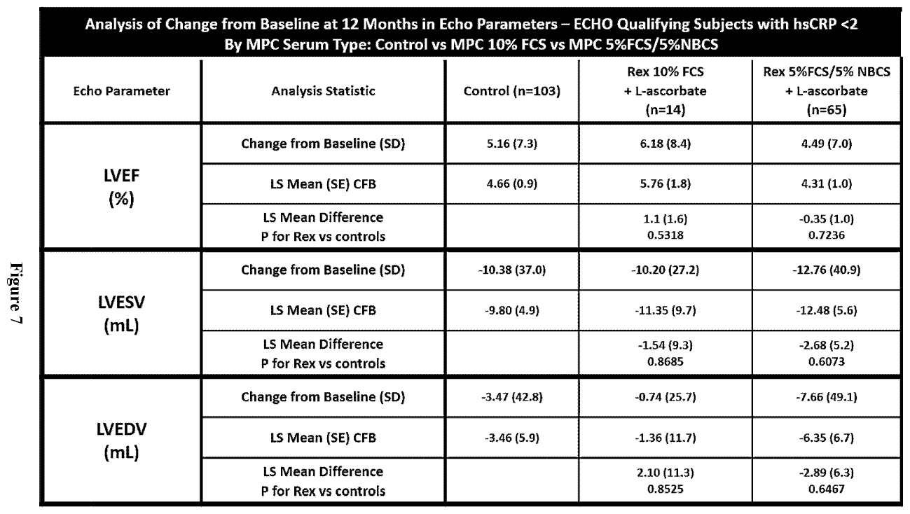

MPCs cultured in media supplemented with newborn serum were also found to reduce other cardiac outcomes in HFrEF patients with CRP>2, including reducing the risk of cardiovascular death by 43% (FIG. 8) and incidence of 3-point MACE (CV Death/MI/Stroke) by 54% (FIG. 9). These data show that MPCs cultured in media supplemented with newborn serum provide improved therapeutic efficacy in the context of persistent inflammation.

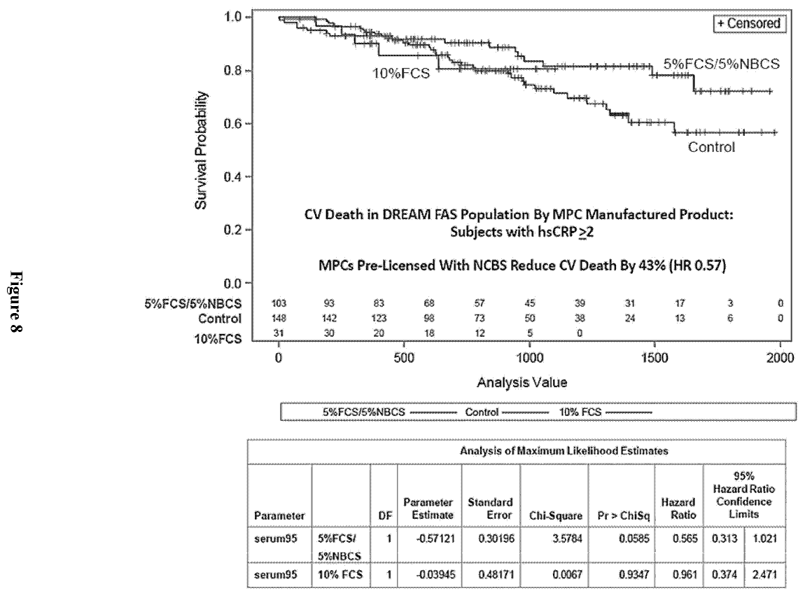

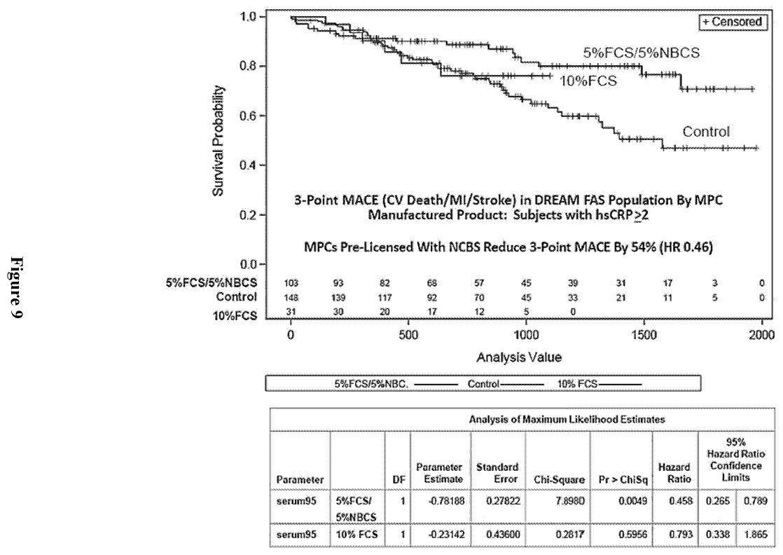

Further analysis of clinical response surprisingly revealed the importance of culture expanding MLPSCs in media supplemented with newborn serum and/or pro-inflammatory cytokines during final passage(s) before administration. MLPSCs cultured in media supplemented with newborn serum and/or pro-inflammatory cytokines during final passages reduced 3-Point MACE (MI, Stroke or CV Death) in patients, irrespective of whether the MLPSCs were culture expanded in FBS during earlier passages. Surprisingly, this reduction in 3-point MACE was observed in all patients, regardless of inflammation status (FIG. 10). Patient sub group analysis revealed that the reduction in 3-point MACE was observed in patients with persistent inflammation (CRP>2 mg/ml;

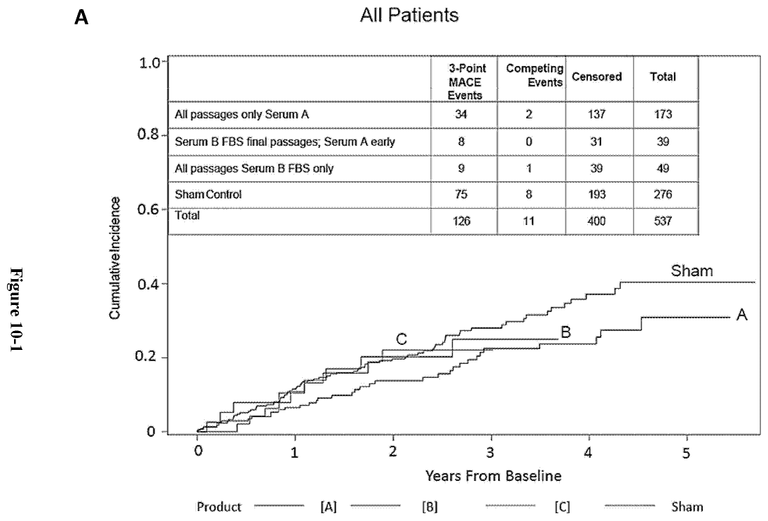

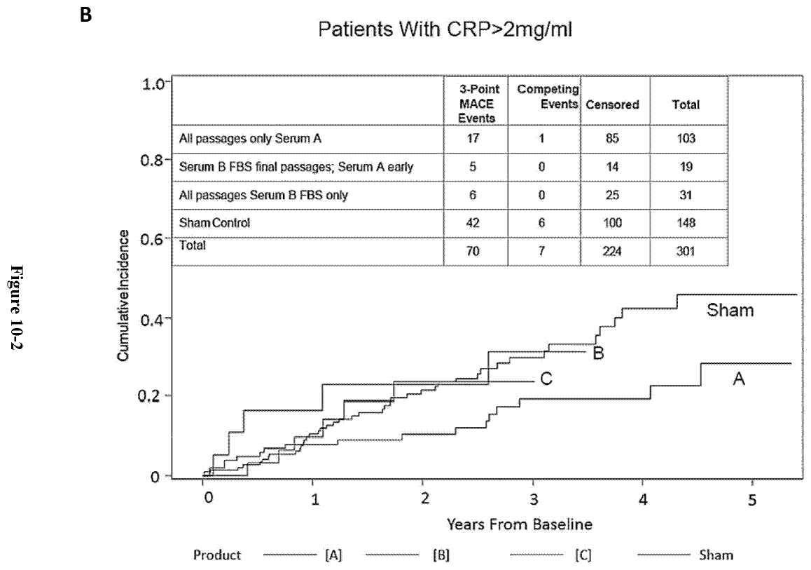

FIG. 10) and, patients in the study at highest risk of Terminal Cardiac Events (TCE; defined as CV Death, Heart Transplant or Left Ventricular Assists Device Implantation). The patients at highest risk of TCE are defined by both CRP>2 mg/ml and NT-proBNP>1000 ng/ml; FIG. 11)).

In contrast, increased adverse events (3-Point MACE) were observed in all patients administered MLPSCs that were culture expanded in media that was not supplemented with newborn serum and/or pro-inflammatory cytokines during final passages before administration. The increase in 3-point MACE was observed regardless of whether the MLPSCs were cultured in media supplemented with newborn serum and/or pro-inflammatory cytokines during earlier passages.

Summary: MPCs expanded in media supplemented with newborn calf serum (NBCS) and/or pro-inflammatory cytokines:
improved left ventricular systolic dysfunction in HFrEF patients with inflammation, as measured by LS mean change in LVEF and LVESV at 12 months;
reduced cardiovascular death by 43% in high-risk NYHA Class II/III patients with HFrEF and inflammation; and,
reduced long-term 3 Point MACE by 54% in high-risk NYHA Class II/III patients with HFrEF and inflammation.

Taken together with the results in Examples 1 and 4, these human trial data indicate that supplementing cell culture media with newborn serum and/or pro-inflammatory cytokines provides a cell population with different functional characteristics, at least in terms of capacity to direct therapeutic efficacy in an inflammatory environment.

These human trial data also evidence the importance of supplementing media with newborn serum and/or pro-inflammatory cytokines during final passage(s), for example, during culture expansion of intermediate cell populations to drug product. Accordingly, the present inventors findings underpin at least two approaches to providing MLPSC populations with improved therapeutic efficacy for administration to patients:
Expansion of MLPSCs to intermediate population in newborn serum and/or pro-inflammatory cytokines to provide cryopreserved intermediate population; expansion of cryopreserved intermediate population in newborn serum and/or pro-inflammatory cytokines to provide drug product.
Expansion of MLPSCs to intermediate population in fetal serum to provide cryopreserved intermediate population; expansion of cryopreserved intermediate population in newborn serum and/or pro-inflammatory cytokines to provide drug product.

Without wishing to be bound by any particular theory, the above referenced data suggest that culturing in media supplemented with newborn serum and/or pro-inflammatory cytokines pre-licenses MPCs to respond more effectively to inflammatory environments. Furthermore, taken together with the results described in Example 4, these data point to a potential mechanism by which MPCs cultured in media supplemented with newborn serum and/or pro-inflammatory cytokines impart improved therapeutic efficacy, namely, the enhanced angiogenesis and increased production of pro-angiogenic growth factors, VEGF, SDF-1α and angiogenin. Accordingly, these data provide basis for a method of selecting cells with a sufficient potency for the treatment of inflammatory disorders. In particular, the data shows that a threshold level of >3.45 ng/mL VEGF, >3000 ng/ml SDF-1α, or >1114 μg/mL angiogenin, with concentrations in advance of these amounts indicating therapeutic potency and increased biological activity of MPCs. For example, cells can be cultured according to the methods disclosed herein, conditioned media could be harvested and measured in the angionesis assay and/or for levels of VEGF and angiogenin. Cells which produce VEGF/angiogenin above the threshold are considered therapeutically potent/biologically active. Similarly, cells which produce conditioned media that enhances angiogenesis as determined by a network area of >0.12 mm²/mm², network length of >5 mm²/mm² and/or branch points of >15 mm²/mm² are also considered to be therapeutically potent/biologically active for treating inflammatory diseases.

The above referenced angiogenic markers may be relevant criteria (in addition to the clinical response criteria shown in heart failure) for characterising the novel MLPSCs produced via culture in cell culture media comprising newborn serum and/or pro-inflammatory cytokines.

### Example 6: MLPSCs Cultured in Media Supplemented with Newborn Fetal Serum are Effective in Treating GvHD

10 GvHD patients were administered (intravenous) MPCs culture expanded with NBCS containing pro-inflammatory cytokines (Examples 2 and 4) once per week at a dose of 2×10⁶ MPCs per kg. Patient response is summarised in Table 6. 80% of GvHD patients administered MPCs cultured in NBCS responded to treatment. 1 patient achieved complete response, 7 patients achieved partial response and 2 patients died.

TABLE 6
GvHD response to MPCs culture expanded with NBCS

| | Non-fetal serum |
|---|---|
| Complete Response (CR) | 1 |
| Partial Response (PR) | 7 |
| Died | 2 |
| No Response (NR) | 0 |
| Total | 10 |

Taken together with the results of at least Example 6, the findings of the present inventors underpin broad application of pre-licensed MLPSCs (e.g. MPCs cultured in the presence of non-fetal serum, in particular newborn serum and/or presence of pro-inflammatory cytokines) for treatment of any disease or disorder characterised by elevated inflammation, in particular diseases characterized by persistent inflammation such as heart failure or T-cell mediated disorders such as GvHD.

### Example 7: Treatment of a HFrEF Patient Population with Licensed MPLSCs

Left ventricular assist devices (LVADs) were implanted in patients with end-stage heart failure with reduced ejection fraction (HFrEF). HFrEF patients who received LVADs were categorized as having either ischemic heart failure or non-ischemic heart failure. LVAD patients in the treatment arm received MPCs cultured in media supplemented with newborn serum or MPCs cultured in media containing 10% FBS. Control patients were not administered stem cell therapy.

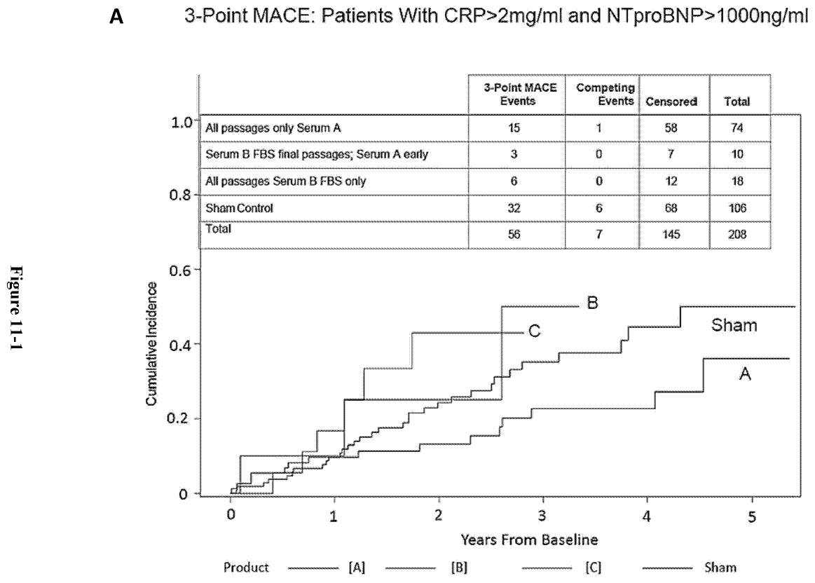

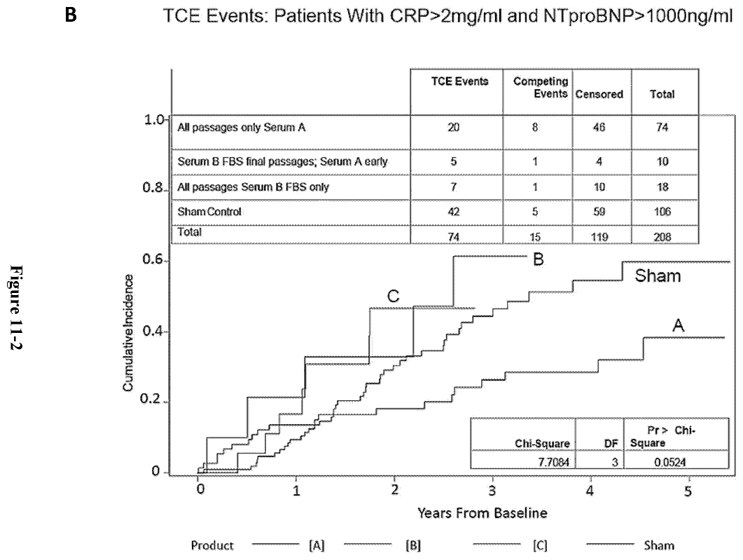

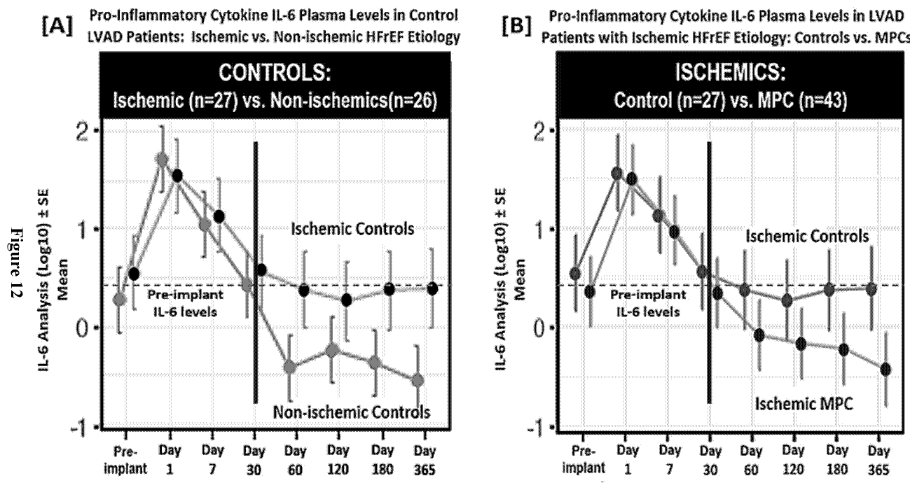

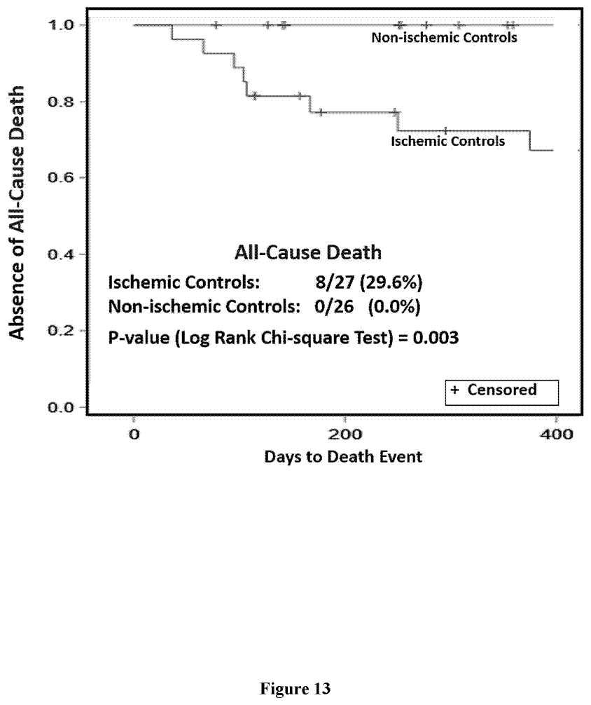

Inflammation was assessed based on serum IL-6 levels. Prior to LVAD implantation, both ischemic and non-ischemic heart failure control patients had similarly elevated IL-6 levels (FIG. 12). From Day 1 through Day 30 post-surgery, similar surgery-related IL-6 increases were observed in both control ischemic and non-ischemic groups (FIG. 12). From Day 30 through Day 365 post-surgery, IL-6 levels in the non-ischemic group reduced to levels below those observed pre-LVAD implantation. However, patients in the ischemic group maintained IL-6 levels similar to the level present pre-LVAD implantation and that were significantly higher than the non-ischemic group (FIG. 12). In addition, the ischemic control group were found to have significantly higher risk for all-cause death over 12 months after LVAD implantation, compared to non-ischemic controls (FIG. 13).

These data suggest that LVAD implantation reduces the inflammatory process associated with end-stage HFrEF in non-ischemic heart failure patients but not in ischemic heart failure patients. Furthermore, LVAD patients with ischemic end-stage HFrEF represent a specific patient subgroup who have persistent inflammation and are at high-risk of all-cause death.

FIG. 12B also shows that administration of MPCs cultured in media supplemented with newborn serum not only reduced IL-6 levels but that the IL-6 levels were, over time, reduced to levels corresponding with non-ischemic control.

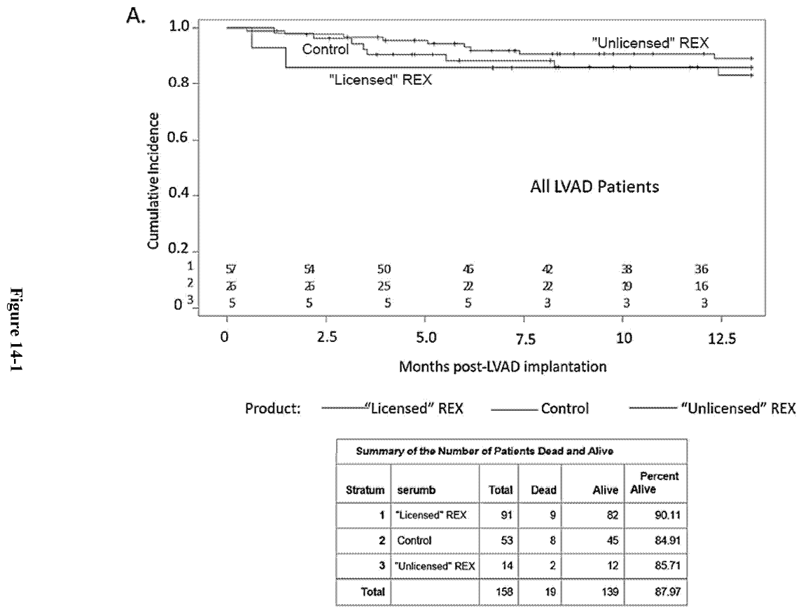

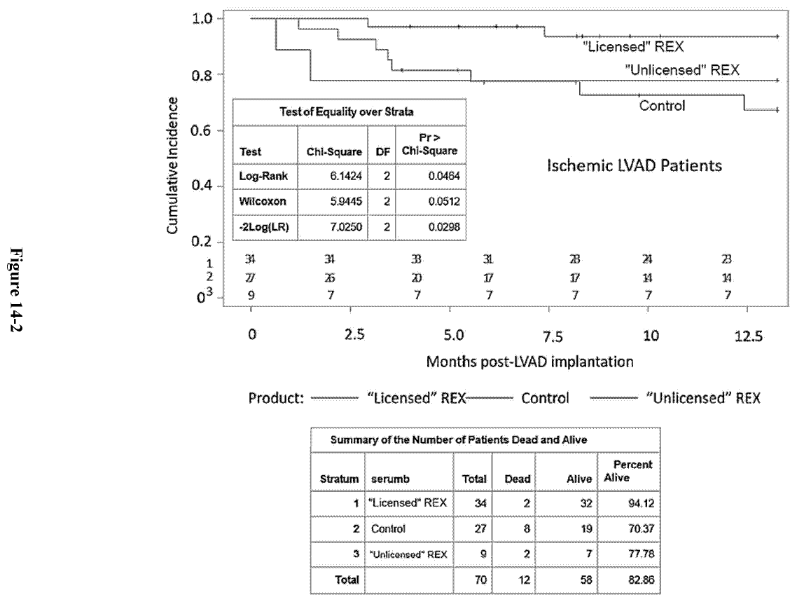

FIGS. 14 and 15 show all-cause death over a period of 12.5 months following LVAD implantation in patients who received MPCs cultured in media supplemented with newborn calf serum, MPCs cultured in media containing 10% FBS, and control patients who did not receive cell therapy. Surprisingly, ischemic LVAD patients who were administered the MPCs cultured in media supplemented with newborn calf serum had significantly reduced all-cause death compared to both the control group and patients who received unlicensed MPCs (FIG. 14B). Specifically, the MPCs cultured in media supplemented with newborn calf serum reduced all-cause death by 83% in the ischemic LVAD patients compared with the ischemic control group.

The findings of the present inventors provide additional evidence (on top of the studies in Example 5) that MPCs culture expanded in media supplemented with at least one pro-inflammatory cytokine and/or newborn calf serum are particularly effective in treating inflammation, in particular treating diseases characterized by persistent inflammation such as heart failure.

### Example 8: Media Analysis and Summary of Findings

Based on the data described in Example 1, cytokine levels were increased in culture medium used to expand MLPSC populations characterised by increase(s) in one or more angiogenic markers (example 4) and increased therapeutic efficacy in both heart failure (example 5) and GvHD (example 6) patients. The correlation between increased pro-inflammatory cytokine levels in culture media and therapeutic efficacy in separate disease indications associated with inflammation suggests a pre-licensing effect on MLPSCs. This is further supported by the reduced all-cause death observed in ischemic LVAD patients (i.e. patients characterised by persistent elevation of IL-6 levels; example 7).

Surprisingly, MLPSCs described herein appear to have been pre-licensed by culture with pro-inflammatory cytokines, despite these cytokines being present at very low levels (e.g. pg/ml levels). This is surprising because it was not previously envisaged that pro-inflammatory cytokines, in particular TNF-alpha and IFN-gamma, could have such dramatic impacts (e.g. increased angiogenic potential; increased therapeutic efficacy in disease indications such as heart failure and GvHD) when present at pg/ml levels. Without wishing to be bound by any particular theory, the data provided by the present inventors, surprisingly suggest synergistic and/or more than additive effects of cytokines in the context of MLPSC culture expansion. For example, the present data indicates that provision of culture medium comprising TNF-alpha and IFN-gamma at concentrations <1 ng/ml can have profound impacts on MLPSCs culture expanded in the same and, that these impacts can be characterised based on levels of various angiogenic markers and/or clinical efficacy in patients.

Accordingly, the present inventors findings represent a significant advance in the art as they have shown how to prepare novel MLPSC populations that can direct improved therapeutic efficacy, in particular in the context of inflammation. These findings not only suggest that improved MLPSC populations can be provided through culture expansion in media supplemented with pro-inflammatory cytokines, they also indicate that relevant pro-inflammatory cytokines can be provided through culture expansion in medium supplemented with newborn serum. Accordingly, the present inventors findings underpin criteria for culture expansion of MLPSC in serum and serum free media.

### Example 9: MLPSC Isolation and Expansion

MLPSCs can be isolated using techniques such as STRO-3+ immunoselection of MPCs or density gradient separation of MSCs.

In general, relevant for bone marrow derived MLPCs, bone marrow (BM) is harvested from healthy normal adult volunteers (20-35 years old). Briefly, 40 ml of BM is aspirated from the posterior iliac crest into lithium-heparin anticoagulant-containing tubes.

BMMNC are prepared by density gradient separation using Lymphoprep (Nycomed Pharma, Oslo, Norway) as previously described (Zannettino et al. 1998). Following centrifugation at 400×g for 30 minutes at 4 C, the buffy layer is removed with a transfer pipette and washed three times in “HHF”, composed of Hank's balanced salt solution (HBSS; Life Technologies, Gaithersburg, MD), containing 5% fetal calf serum (FCS, CSL Limited, Victoria, Australia).

Relevant for immunoselection, STRO-3+ (or TNAP+) cells are subsequently isolated by magnetic activated cell sorting as previously described (Gronthos et al. 2003; Gronthos and Simmons 1995). Briefly, approximately 1-3×108 BMMNC are incubated in blocking buffer, consisting of 10% (v/v) normal rabbit serum in HHF for 20 minutes on ice. The cells are incubated with 200 ul of a 10 ug/ml solution of STRO-3 mAb in blocking buffer for 1 hour on ice. The cells are subsequently washed twice in HHF by centrifugation at 400×g. A 1/50 dilution of goat anti-mouse-biotin (Southern Biotechnology Associates, Birmingham, UK) in HHF buffer is added and the cells incubated for 1 hour on ice. Cells are washed twice in MACS buffer (Ca2+- and Mn2+-free PBS supplemented with 1% BSA, 5 mM EDTA and 0.01% sodium azide) as above and resuspended in a final volume of 0.9 ml MACS buffer.

One hundred ul streptavidin microbeads (Miltenyi Biotec; Bergisch Gladbach, Germany) are added to the cell suspension and incubated on ice for 15 minutes. The cell suspension is washed twice and resuspended in 0.5 ml of MACS buffer and subsequently loaded onto a mini MACS column (MS Columns, Miltenyi Biotec), and washed three times with 0.5 ml MACS buffer to retrieve the cells which did not bind the STRO-3 mAb (deposited on 19 Dec. 2005 with American Type Culture Collection (ATCC) under accession number PTA-7282-see International Publication No. WO 2006/108229). After addition of a further 1 ml MACS buffer, the column is removed from the magnet and the TNAP+ cells are isolated by positive pressure. An aliquot of cells from each fraction can be stained with streptavidin-FITC and the purity assessed by flow cytometry.

Alternatively, MSCs may be expanded from BMMNC using plastic adherence techniques. For example, bone marrow mononuclear cells can be isolated using ficoll-hypaque and placed into two T175 flasks with 50 ml per flask of culture expansion medium which includes alpha modified MEM (αMEM) containing gentamycin, glutamine (2 mM) and 10% (v/v) fetal bovine serum (FBS).

Cells are cultured for 2-3 days in 37° C., 5% CO₂ at which time the non-adherent cells are removed; the remaining adherent cells are continually cultured until cell confluence reaches 70% or higher (7-10 days), and then the cells are trypsinized and replaced in six T175 flasks with expansion medium.

It will be appreciated by persons skilled in the art that numerous variations and/or modifications may be made to the disclosure as shown in the specific embodiments without departing from the spirit or scope of the disclosure as broadly described. The present embodiments are, therefore, to be considered in all respects as illustrative and not restrictive.

The present application claims priority from U.S. 63/386,876 filed 9 Dec. 2022, U.S. 63/386,883 filed 9 Dec. 2022 and U.S. 63/507,009 filed 8 Jun. 2023, the disclosures of which are incorporated herein by reference.

All publications discussed and/or referenced herein are incorporated herein in their entirety.

Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

1. A method of treating an inflammatory disease in a subject, the method comprising administering to the subject a composition comprising a population of culture expanded mesenchymal lineage precursor or stem cells (MLPSCs) or extracellular vesicles obtained therefrom, wherein the MLPSCs have been culture expanded in a cell culture medium comprising at least one pro-inflammatory cytokine, and wherein the subject has persistent inflammation characterized by C-reactive protein (CRP) levels ≥2 mg/L.

2. The method according to claim 1, wherein the cell culture medium comprises:
IFN-gamma and/or TNF-alpha; and/or,
one or more pro-inflammatory cytokines selected from the group consisting of IL-6; IL-8; IL-17A; MCP-1; MIP-1-alpha; MIP-1-beta; IP-10.

3. The method according to claim 1, wherein the cell culture medium three or more pro-inflammatory cytokines.

4. The method according to claim 1, wherein the cell culture medium contains two or more pro-inflammatory cytokines selected from the group consisting of IL-6; IL-8; IL-17A; MCP-1; MIP-1-alpha; MIP-1-beta; IP-10.

5-7. (canceled)

8. The method according to claim 1, wherein the level of IFN-gamma is <1 ng/ml, <500 pg/ml, or <100 pg/ml; and/or wherein the level of TNF-alpha is <1 ng/ml, <750 pg/ml, or <400 pg/ml.

9. (canceled)

10. The method according to claim 1, wherein the cell culture medium serum which comprises the pro-inflammatory cytokines.

11. The method according to claim 10, wherein the cell culture medium comprises a non-fetal serum.

12. The method according to claim 10, wherein
the serum is a newborn mammalian serum;
the serum is newborn calf serum (NBCS);
the serum is obtained no more than 21 days after birth;
the serum is obtained between the day of birth and 21 days after birth; between the day of birth and 14 days after birth; between the day of birth and 10 days after birth; or, between the day of birth and 7 days after birth; or
the serum is obtained between the day of birth and 10 days after birth.

13-16. (canceled)

17. The method according to claim 10, wherein the cell culture medium is characterized by one or more or all of the following:
i. a level of IFN-gamma greater than 1 pg/ml;
ii a level of TNF-alpha greater than 2 pg/ml;
iii. a level of IL-6 greater than 3 pg/ml;
iv. a level of IL-8 greater than 500 pg/ml;
V. a level of IL-17A greater than 0.2 pg/ml;
vi. a level of MCP-1 greater than 3 pg/ml;
vii. a level of MIP-1-alpha greater than 0.5 pg/ml;
viii. a level of MIP-1-beta greater than 3 pg/ml; and
ix. a level of IP-10 greater than 500 pg/ml.

18. The method according to claim 10, wherein the cell culture medium comprises at least 5% (v/v) newborn calf serum or 5% (v/v) non-fetal serum.

19-20. (canceled)

21. The method according to claim 10, wherein the cell culture medium comprises 5% non-fetal serum and 5% fetal serum.

22. (canceled)

23. The method according to claim 1, wherein the inflammatory disease is mediated by T-cell activation.

24. The method of claim 23, wherein the inflammatory disease is refractory to steroid immunosuppressant and/or ruxolitinib.

25-28. (canceled)

29. The method according to claim 1, wherein the inflammatory disease is selected from the group consisting of graft versus host disease (GvHD), pruritus, skin inflammation, psoriasis, multiple sclerosis, rheumatoid arthritis, osteoarthritis, systemic lupus erythematosus, Hashimoto's thyroidis, myasthenia gravis, type I or II diabetes, an associated condition or symptom of diabetes, diabetic nephropathy, asthma, inflammatory lung injury, inflammatory liver injury, inflammatory glomerular injury, atopic dermatitis, allergic contact dermatitis, irritant contact dermatitis, seborrhoeic dermatitis, Sjoegren's syndrome, keratoconjunctivitis, uveitis, inflammatory bowel disease, Crohn's disease, ulcerative colitis, an inflammatory disease of the joints, skin, or muscle, acute or chronic idiopathic inflammatory arthritis, myositis, a demyelinating disease, chronic obstructive pulmonary disease (COPD), interstitial lung disease, interstitial nephritis chronic active hepatitis, multi-system inflammatory syndrome (MIS), acute respiratory distress syndrome (ARDS), and hyperinflammation.

30-33. (canceled)

34. The method according to claim 1, wherein the inflammatory disease is lower back pain, wherein the lower back pain is associated with a degenerated disc.

35. The method according to claim 1, wherein the MLPSCs or extracellular vesicles are administered systemically or intravenously, and/or wherein the MLPSCs or extracellular vesicles are administered over multiple doses.

36-37. (canceled)

38. The method according to claim 1, wherein the mesenchymal lineage precursor or stem cells are mesenchymal precursor cells (MPCs).

39. (canceled)

40. The method according to claim 1, wherein the mesenchymal lineage precursor or stem cells are mesenchymal stem cells (MSCs).

41. The method according to claim 1, wherein the MLPSCs are allogeneic, wherein the cells have been cryopreserved prior to administration, and/or wherein the cells have been cryopreserved prior to culture expansion in a cell culture medium comprising non-fetal serum.

42-43. (canceled)

44. The method according to claim 1 which comprises administering between 1×10⁷ and 2×10⁸ cells.

45-51. (canceled)
